SEQUENCE LISTING (6 sequences)

SEQ ID NO: 1 - DNA, 2191 bp, Artificial Sequence
  human nAChR alpha4
tgcttgttctttttgcagaagctcagaataaacgctcaactttggcagatccatggagctagggggccccggagcgccgcggctgctgccgccgctgctgctgcttctggggaccggcctcctgcgcgccagcagccatgtggagacccgggcccacgccgaggagcggctcctgaagaaactcttctccggttacaacaagtggtcccgacccgtggccaacatctcggacgtggtcctcgtccgcttcggcctgtccatcgctcagctcattgacgtggatgagaagaaccagatgatgaccacgaacgtatgggtgaagcaggagtggcacgactacaagctgcgctgggacccagctgactatgagaatgtcacctccatccgcatcccctccgagctcatctggcggccggacatcgtcctctacaacaatgctgacggggacttcgcggtcacccacctgaccaaggcccacctgttccatgacgggcgggtgcagtggactcccccggccatttacaagagctcctgcagcatcgacgtcaccttcttccccttcgaccagcagaactgcaccatgaaattcggctcctggacctacgacaaggccaagatcgacctggtgaacatgcacagccgcgtggaccagctggacttctgggagagtggcgagtgggtcatcgtggacgccgtgggcacctacaacaccaggaagtacgagtgctgcgccgagatctacccggacatcacctatgccttcgtcatccggcggctgccgctcttctacaccatcaacctcatcatcccctgcctgctcatctcctgcctcaccgtgctggtcttctacctgccctccgagtgcggcgagaagatcacgctgtgcatctccgtgctgctgtcgctcaccgtcttcctgctgctcatcaccgagatcatcccgtccacctcactggtcatcccactcatcggcgagtacctgctgttcaccatgatcttcgtcaccctgtccatcgtcatcacggtcttcgtgctcaacgtgcaccaccgctcgccacgcacgcacaccatgcccacctgggtacgcagggtcttcctggacatcgtgccacgcctgctcctcatgaagcggccgtccgtggtcaaggacaattgccggcggctcatcgagtccatgcataagatggccagtgccccgcgcttctggcccgagccagaaggggagccccctgccacgagcggcacccagagcctgcaccctccctcaccgtccttctgcgtccccctggatgtgccggctgagcctgggccttcctgcaagtcaccctccgaccagctccctcctcagcagcccctggaagctgagaaagccagcccccacccctcgcctggaccctgccgcccgtcccacggcacccaggcaccagggctggccaaagccaggtccctcagcgtccagcacatgtccagccctggcgaagcggtggaaggcggcgtccggtgccggtctcggagcatccagtactgtgttccccgagacgatgccgcccccgaggcagatggccaggctgccggcgccctggcctctcgcaacacccactcggctgagctcccacccccagaccagccctctccgtgcaaatgcacatgcaagaaggagccctcttcggtgtccccgagtgccacggtcaagacccgcagcaccaaagcaccgcccccgcacctgcccctgtcgccggccctgacccgggcggtggagggcgtccagtacattgcagaccacctgaaggccgaagacacagacttctcggtgaaggaggactggaagtacgtggccatggtcatcgaccgcatcttcctctggatgttcatcatcgtctgcctgctggggacggtgggcctcttcctgccgccctggctggctggcatgatctaggaagggaccgggagccgcgtggcctggggctgccgtgcacggggccagcatttggttaccantaaaccagcctcaagaacacccgaatggggtntttaaggtacataatnccaanttacantttacaaaatgttgtcccccaaaatgtagccatttgtatntgntcctaataaaaagaaagttttttcacattttaaaaaaaaaaaaaaaaaaaaaaaaaaaaaaaaacccccccccccccccccctgcaggtcg

SEQ ID NO: 2 - DNA, 1537 bp, Artificial Sequence
  human nAChR beta2
gatctgcccgcggcatggcccggcgctgcggccccgtggcgctgctccttggcttcggcctcctccggctgtgctcaggggtgtggggtgcggatacagaggagcggctggtggagcatctcctggatccttcccgctacaacaagcttatccgcccagccaccaatggctctgagctggtgacagtacagcttatggtgtcactggcccagctcatcagtgtgcatgagcgggagcagatcatgaccaccaatgtctggctgacccaggagtgggaagattatcgcctcacctggaagcctgaagagtttgacaacatgaagaaagttcggctcccttccaaacacatctggctcccagatgtggtcctgtacaacaatgctgacggcatgtacgaggtgtccttctattccaatgccgtggtctcctatgatggcagcatcttctggctgccgcctgccatctacaagagcgcatgcaagattgaagtaaagcacttcccatttgaccagcagaactgcaccatgaagctccgttcgtggacctacgaccgcacagagatcgacttggtgctgaagagtgaggtggccagcctagacgacttcacacctagtggtgagtgggacatcgtggcgctgccgggccggcgcaacgagaaccccgacgactctacgtacgtggacatcacgtatgacttcatcattcgccgcaagccgctcttctacaccatcaacctcatcatcccctgtgtgctcatcacctcgctagccatccttgccttctacctgccatccgactgtggcgagaagatgacgttgtgcatctcagtgctgctggcgctcacggtcttcctgctgctcatctccaagatcgtgcctcccacctccctcgacgtgccgctcgtcggcaagtacctcatgttcaccatggtgcttgtcaccttctccatcgtcaccagcgtgtgcgtgctcaacgtgcaccaccgctcgcccaccacgcacaccatggcgccctgggtgaaggtcgtcttcctggagaagctgcccgcgctgctcttcatgcagcagccacgccatcattgcgcccgtcagcgcctgcgcctgcggcgacgccagcgtgagcgcgagggcgctggagccctcttcttccgcgaagccccaggggccgactcctgcacgtgcttcgtcaaccgcgcgtcggtgcaggggttggccggggccttcggggctgagcctgcaccagtggcgggccccgggcgctcaggggagccgtgtggctgtggcctccgggaggcggtggacggcgtgcgtttcatcgcagaccacatgcggagcgaggacgatgaccagagcgtgagtgtggactggaagtacgtcgccatggtgatcgaccgcctcttcctctggatctttgtctttgtctgtgtctttggcaccatcggcatgttcctgcagcctctcttccagaactacaccaccaccaccttcctccactcagaccactcagcccccagctccaagtgaggcccttccagatc

SEQ ID NO: 3 - DNA, 1695 bp, Artificial Sequence
  human nAChR alpha6
agtgggcttctgatgatgtcaaggttggatgcatgtggctgactgatagctctttgttttccacaatcctttgcctaggaaaaaggaatccaagtgtgttttaaccatgctgaccagcaaggggcagggattccttcatgggggcttgtgtctctggctgtgtgtgttcacacctttctttaaaggctgtgtgggctgtgcaactgaggagaggctcttccacaaactgttttctcattacaaccagttcatcaggcctgtggaaaacgtttccgaccctgtcacggtacactttgaagtggccatcacccagctggccaacgtggatgaagtaaaccagatcatggaaaccaatttgtggctgcgtcacatctggaatgattataaattgcgctgggatccaatggaatatgatggcattgagactcttcgcgttcctgcagataagatttggaagcccgacattgttctctataacaatgctgttggtgacttccaagtagaaggcaaaacaaaagctcttcttaaatacaatggcatgataacctggactccaccagctatttttaagagttcctgccctatggatatcacctttttcccttttgatcatcaaaactgttccctaaaatttggttcctggacgtatgacaaagctgaaattgatcttctaatcattggatcaaaagtggatatgaatgatttttgggaaaacagtgaatgggaaatcattgatgcctctggctacaaacatgacatcaaatacaactgttgtgaagagatatacacagatataacctattctttctacattagaagattgccgatgttttacacgattaatctgatcatcccttgtctctttatttcatttctaaccgtgttggtcttttaccttccttcggactgtggtgaaaaagtgacgctttgtatttcagtcctgctttctctgactgtgtttttgctggtcatcacagaaaccatcccatccacatctctggtggtcccactggtgggtgagtacctgctgttcaccatgatctttgtcacactgtccatcgtggtgactgtgtttgtgttgaacatacactaccgcaccccaaccacgcacacaatgcccaggtgggtgaagacagttttcctgaagctgctgccccaggtcctgctgatgaggtggcctctggacaagacaaggggcacaggctctgatgcagtgcccagaggccttgccaggaggcctgccaaaggcaagcttgcaagccatggggaacccagacatcttaaagaatgcttccattgtcacaaatcaaatgagcttgccacaagcaagagaagattaagtcatcagccattacagtgggtggtggaaaattcggagcactcgcctgaagttgaagatgtgattaacagtgttcagttcatagcagaaaacatgaagagccacaatgaaaccaaggaggtagaagatgactggaaatacgtggccatggtggtggacagagtatttctttgggtatttataattgtctgtgtatttggaactgcagggctatttctacagccactacttgggaacacaggaaaatcttaaaatgtattttcttttatgttcagaaatttacagacaccatatttgttctgcattccctgccacaaggaaaggaaagcaaaggcttcccacccaagtcccccatc

SEQ ID NO: 4 - DNA, 2462 bp, Artificial Sequence
  human nAChR beta4
aattcggcacgagccgccagcaaacctcgggggccaggaccggcgctcactcgaccgcgcggctcacgggtgccctgtgaccccacagcggagctcgcggcggctgccacccggccccgccggccatgaggcgcgcgccttccctggtccttttcttcctggtcgccctttgcgggcgcgggaactgccgcgtggccaatgcggaggaaaagctgatggacgaccttctgaacaaaacccgttacaataacctgatccgcccagccaccagctcctcacagctcatctccatcaagctgcagctctccctggcccagcttatcagcgtgaatgagcgagagcagatcatgaccaccaatgtctggctgaaacaggaatggactgattaccgcctgacctggaacagctcccgctacgagggtgtgaacatcctgaggatccctgcaaagcgcatctggttgcctgacatcgtgctttacaacaacgccgacgggacctatgaggtgtctgtctacaccaacttgatagtccggtccaacggcagcgtcctgtggctgccccctgccatctacaagagcgcctgcaagattgaggtgaagtactttcccttcgaccagcagaactgcaccctcaagttccgctcctggacctatgaccacacggagatagacatggtcctcatgacgcccacagccagcatggatgactttactcccagtggtgagtgggacatagtggccctcccagggagaaggacagtgaacccacaagaccccagctacgtggacgtgacttacgacttcatcatcaagcgcaagcctctgttctacaccatcaacctcatcatcccctgcgtgctcaccaccttgctggccatcctcgtcttctacctgccatccgactgcggcgagaagatgacactgtgcatctcagtgctgctggcactgacattcttcctgctgctcatctccaagatcgtgccacccacctccctcgatgtgcctctcatcggcaagtacctcatgttcaccatggtgctggtcaccttctccatcgtcaccagcgtctgtgtgctcaatgtgcaccaccgctcgcccagcacccacaccatggcaccctgggtcaagcgctgcttcctgcacaagctgcctaccttcctcttcatgaagcgccctggccccgacagcagcccggccagagccttcccgcccagcaagtcatgcgtgaccaagcccgaggccaccgccacctccaccagcccctccaacttctatgggaactccatgtactttgtgaaccccgcctctgcagcttccaagtctccagccggctctaccccggtggctatccccagggatttctggctgcggtcctctgggaggttccgacaggatgtgcaggaggcattagaaggtgtcagcttcatcgcccagcacatgaagaatgacgatgaagaccagagtgtcgttgaggactggaagtacgtggctatggtggtggaccggctgttcctgtgggtgttcatgtttgtgtgcgtcctgggcactgtggggctcttcctaccgcccctcttccagacccatgcagcttctgaggggccctacgctgcccagcgtgactgagggccccctgggttgtggggtgagaggatgtgagtggccgggtgggcactttgctgcttctttctgggttgtggccgatgaggccctaagtaaatatgtgagcattggccatcaaccccatcaaaccagccacagccgtggaacaggcaaggatgggggcctgggctgtcctctctgaatgccttggagggatcccaggaagccccagtaggagggagcttcagacagttcaattctggcctgtcttccttccctgcaccgggcaatggggataaagatgacttcgtagcagcacctactatgcttcaggcatggtgccggcctgcctctccatcaccatctctctccacttccccttgtccagttcctcacacacttctagattcttcccagctcagaggcttggcatttgccatatcatcatctttttttttttcttttaaacggagtcttgctatatcgcccaggctcaagtgattctcctgcctcaacctcccaagtagctgggattacagaaagccgccaccgtgcccagctaatttttgtatttttgttggccaggctgatttcgaactcctgacctcagatgacccacctgcctcggcctcccaaagtgctgggattacaggtgtgagccactatgcctggccttccctatcttctacctgaaccttcttcctcttcccccagggcttcccaagctctttccacagccacatcagtcagggcatggctccaatccattctttgctccaatgtcacctcctctgagaggccttccctaaccacccaatcattctaaagcagcctccctacagtcacgttaccctgcctccttcc

SEQ ID NO: 5 - DNA, 1551 bp, Artificial Sequence
  human nAChR beta3
aacccccttttccagtggaaatgctctgttgttaaaaaggaagaaactgtctttctgaaactgacatcacgatgctcccagattttatgctggttctcatcgtccttggcatcccttcctcagccaccacaggtttcaactcaatcgccgaaaatgaagatgccctcctcagacatttgttccaaggttatcagaaatgggtccgccctgtattacattctaatgacaccataaaagtatattttggattgaaaatatcccagcttgtagatgtggatgaaaagaatcagctgatgacaaccaatgtgtggctcaaacaggaatggacagaccacaagttacgctggaatcctgatgattatggtgggatccattccattaaagttccatcagaatctctgtggcttcctgacatagttctctttgaaaatgctgacggccgcttcgaaggctccctgatgaccaaggtcatcgtgaaatcaaacggaactgttgtctggacccctcccgccagctacaaaagctcctgcaccatggacgtcacgtttttcccgttcgaccgacagaactgctccatgaagtttggatcctggacttatgatggcaccatggttgacctcattttgatcaatgaaaatgtcgacagaaaagacttcttcgataacggagaatgggaaatactgaatgcaaaggggatgaaggggaacagaagggacggcgtgtactcctatccctttatcacgtattccttcgtcctgagacgcctgcctttattctataccctctttctcatcatcccctgcctggggctgtctttcctaacagttcttgtgttctatttaccttcggatgaaggagaaaaactttcattatccacatcggtcttggtttctctgacagttttccttttagtgattgaagaaatcatcccatcgtcttccaaagtcattcctctcattggagagtacctgctgttcatcatgatttttgtgaccctgtccatcattgttaccgtgtttgtcattaacgttcaccacagatcttcttccacgtaccaccccatggccccctgggttaagaggctctttctgcagaaacttccaaaattactttgcatgaaagatcatgtggatcgctactcatccccagagaaagaggagagtcaaccagtagtgaaaggcaaagtcctcgaaaaaaagaaacagaaacagcttagtgatggagaaaaagttctagttgcttttttggaaaaagctgctgattccattagatacatttccagacatgtgaagaaagaacattttatcagccaggtagtacaagactggaaatttgtagctcaagttcttgaccgaatcttcctgtggctctttctgatagtgtcagtaacaggctcggttctgatttttacccctgctttgaagatgtggctacatagttaccattaggaatttaaaagacataagactaaattacaccttagacctgacatctggctatcacacagacagaatccaaatgcatgtgcttgttctacgaaccccgaatgcg

SEQ ID NO: 6 - DNA, 1664 bp, Artificial Sequence
  human nAChR alpha5
attccgggagctgtggcgcggagcggcccctctgctgcgtctgccctcgttttgtctcacgactcacactcagtgctccattccccaagagttcgcgttccccgcgcggcggtcgagaggcggctgcccgcggtcccgcgcgggcgcggggcgatggcggcgcgggggtcagggccccgcgcgctccgcctgctgctcttggtccagctggtcgcggggcgctgcggtctagcgggcgcggcgggcggcgcgcagagaggattatctgaaccttcttctattgcaaaacatgaagatagtttgcttaaggatttatttcaagactacgaaagatgggttcgtcctgtggaacacctgaatgacaaaataaaaataaaatttggacttgcaatatctcaattggtggatgtggatgagaaaaatcagttaatgacaacaaacgtctggttgaaacaggaatggatagatgtaaaattaagatggaaccctgatgactatggtggaataaaagttatacgtgttccttcagactctgtctggacaccagacatcgttttgtttgataatgcagatggacgttttgaagggaccagtacgaaaacagtcatcaggtacaatggcactgtcacctggactccaccggcaaactacaaaagttcctgtaccatagatgtcacgtttttcccatttgaccttcagaactgttccatgaaatttggttcttggacttatgatggatcacaggttgatataattctagaggaccaagatgtagacaagagagatttttttgataatggagaatgggagattgtgagtgcaacagggagcaaaggaaacagaaccgacagctgttgctggtatccgtatgtcacttactcatttgtaatcaagcgcctgcctctcttttataccttgttccttataataccctgtattgggctctcatttttaactgtacttgtcttctatcttccttcaaatgaaggtgaaaagatttgtctctgcacttcagtacttgtgtctttgactgtcttccttctggttattgaagagatcataccatcatcttcaaaagtcatacctctaattggagagtatctggtatttaccatgatttttgtgacactgtcaattatggtaaccgtcttcgctatcaacattcatcatcgttcttcctcaacacataatgccatggcgcctttggtccgcaagatatttcttcacacgcttcccaaactgctttgcatgagaagtcatgtagacaggtacttcactcagaaagaggaaactgagagtggtagtggaccaaaatcttctagaaacacattggaagctgcgctcgattctattcgctacattacaacacacatcatgaaggaaaatgatgtccgtgaggttgttgaagattggaaattcatagcccaggttcttgatcggatgtttctgtggacttttcttttcgtttcaattgttggatctcttgggctttttgttcctgttatttataaatgggcaaatatattaataccagttcatattggaaatgcaaataagtgaagcctcccaagggactgaagtatacatttagttaacacacatatatctgatggcacctataaaattatgaaaatgtaagttatgtgttaaatttagtgcaagct

(19) United States
(12) Patent Application Publication
(10) Pub. No.: US 2009/0068642 A1
(43) Pub. Date: Mar. 12, 2009

(54) Composition and pharmacology of novel alpha6-containing nicotinic acetylcholine receptors

(76) Inventors: Merouane Bencherif, Winston Salem, NC (US); Ronald J. Lukas, Phoenix, AZ (US); Sharon Rae Letchworth, Kernersville, NC (US); Vladimir Grinevich, Kernersville, NC (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 11/387,619

(22) Filed: Mar. 23, 2006

Related U.S. Application Data
(63) Continuation of application No. PCT/US04/31615, filed on Sep. 24, 2004.
(60) Provisional application No. 60/505,966, filed on Sep. 24, 2003.

(51) Int. Cl. C12Q 1/68; C12N 5/08

(52) U.S. Cl. 435/6; 435/366

(57) ABSTRACT

Nicotinic acetylcholine receptors (nAChRs) comprising the α6 receptor subunit; nucleic acids, including vectors, comprising subunit incoding sequences; cells expressing the nAChRs of the invention; and methods of screening compounds are provided.

This application is a continuation of International Application No. PCT/US04/31615 filed Sep. 24, 2004, fully incorporated herein by reference, itself claiming benefit of U.S. Provisional Patent Application No. 60/505,966, filed Sep. 24, 2003.

### BACKGROUND OF THE INVENTION

The nicotinic acetylcholinergic receptor (nAChR) subunit, α6, is found in brain regions that contain neurons expressing dopamine (DA) and norepinephrine (NE), as well as in retinal neurons. This observation indicates that α6-containing (α6*) nAChR receptors can be relevant to medical indications where these neurons degenerate or malfunction, such as Parkinson's disease, Lewy Body dementia, supranuclear palsy, substance abuse, attentional deficits, retinal degeneration, and disorders of sensory integration. To facilitate rapid development of therapeutics for these indications, an in vitro model of α6 pharmacology is desirable.

Nicotinic receptors are known to modulate striatal DA release. Two subtypes of nAChRs located on striatal dopaminergic terminals are thought to be involved: α4β2 and α6* nAChR (Zoli et al., 2002). While the pharmacology of the α4β2 nAChR subtype is well characterized, much less is known about α6* nAChR. αCTxMII is a 16 amino acid peptide with two disulfide bonds (Cartier et al., 1996) that binds to α6-containing nAChR (Champtiaux et al., 2002). αCtxMII is an antagonist and partially blocks nicotine-stimulated dopamine release in synaptosomes prepared from rat striatum (Kulak et al, 1997). However, studies using the radiolabeled form of αCTxMII to identify α6* nAChR have proven impractical because of peptide instability and high levels of non-specific binding.

Due to the wide distribution and diversity of nAChR in the CNS, there are no regions in the brain solely expressing α6-containing receptors. Furthermore, no immortalized cell lines that naturally express α6* nAChR have been identified. Therefore, there is a need to develop a tissue source that produces an isolated population of α6-containing receptors for detailed study.

The α6 subunit, in combination with other nAChR subunits, has been expressed in oocytes to examine the function of these receptors by electrophysiology (Gerzanich et al., 1997; Kuryatov et al., 2000). However, the oocyte cell system does not allow for continuous passage of the cells as a tissue source for the receptor. In continuous cell lines, the coexpression of α6 with β2, β4, or α3/β4 has been described (Fucile et al., 1998) and a chimeric α6/α4 subunit coexpressed with β4 in HEK-293 cells has recently been reported (Evans et al., 2003).

Messenger RNA for the α6 subunit is expressed in the substantia nigra/ventral tegmental area and in the locus coeruleus, brain regions that contain dopaminergic and noradrenergic neurons, respectively (Le Novere et al., 1996). Besides α6 mRNA, these regions also produce α4, α5, β2, β3, and β4 mRNA (Quik et al., 2000; Charpantier et al., 1998). Furthermore, α6 and β3 mRNAs have been shown to co-localize in multiple areas (Le Novere et al., 1996). The precise subunit combination of α6-containing nAChR in brain is not known, and it is possible that several combinations of nAChR exist.

### SUMMARY OF THE INVENTION

In one aspect of the present invention, the α6 subunit (α6*-nAChR) is heterologously expressed in human SH-EP 1 epithelial cells. Nicotinic acetylcholine receptors (nAChRs) exist as a diverse family of subtypes composed of different subunit combinations. nAChRs are thought to be the principal targets involved in nicotine dependence. Although nAChR α6 subunits are not abundant in the mammalian brain, message encoding them is enriched in dopaminergic brain centers implicated in reward including the ventral tegmental area and nucleus accumbens. However, little is known about other nAChR subunits serving as assembly partners with the α6 subunit or about α6*-nAChR pharmacology and function. Therefore, a series of stably transfected cell lines was generated based on the human SH-EP1 epithelial host heterologously expressing human α6 and other subunits in binary, ternary, or quaternary combinations. The ⁸⁶Rb⁺ efflux assay was used to assess α6*-nAChR function in transfected cells. Pharmacologically distinct, functional nAChR are formed from cells transfected with: α4 and β2 subunits; α6, α4, β2 and β3 subunits; α6, β4, β3 and α5 subunits. Absolute levels of function for quaternary complexes containing the α6 subunit but lacking the α4 subunit are lower than functional levels for α4β2-nAChR or for quaternary complexes containing α6 and α4 subunits. For the latter, co-assembly of α6 and α4 subunits is indicated by tandem immunoprecipitation-Western blot analyses. Thus, nAChR receptor subtype combinations having useful pharmacological characteristics are provided. These can be stably transfected in cell lines which are useful reagents for screening of compounds useful for treating diseases associated with α6-nAChR mediated activity. The receptor subunit combinations of the invention are also useful for study of α6*-nAChR and for elucidation of roles played by α6*-nAChR in nicotine dependence and nicotinic cholinergic signaling. The pharmacology of α6-containing nicotinic receptors stable expressed in SH-EP1 cells has been examined in order to identify subunit combinations useful in screening for compounds useful, or more likely to be useful, for the diagnosis or treatment of disease.

Nicotinic acetylcholine receptors are known to modulate dopamine release from striatal terminals, suggesting therapeutic potential for Parkinson's disease. Messenger RNA for α6 subunit is robustly expressed by dopamine neurons (Le Novere et al., 1996), and α6 protein has been isolated from striatal terminals (Zoli et al., 2002). Furthermore, α6 binding sites are depleted in MPTP models (Quik et al. 2001). As one aspect of the present invention, α6-containing receptors were characterized using cells of the SH-EP1 human epithelial line stably transfected with the α6 nicotinic receptor subunit in combination with α4, α5, β2, β3 or β4 subunits. [³H]-epibatidine (EPI) was used to define receptor binding, whereas ⁸⁶Rb⁺ efflux was used to detect functional responses. Cells transfected with both α6 and α4 genes in combination with other subunits exhibited α4-like pharmacological profiles. For example, cells expressing α4β2, α6α4β2, or α6α4β2β3 combinations exhibited similar profiles, and cells expressing α4β4, α6α4β4, α6α4β4β3, or α6α4β4α5 produced nearly identical profiles, tentatively suggesting no or minimal contributions of α6, β3 and/or α5 subunits. Cells expressing the α6β4β3 combination did not exhibit detectable [³H]-EPI binding (or function), but those expressing α6β4β3α5 did (K_{D}=70 μM; Bₘₐₓ=41 fmol/mg). The rank order of potency for nicotinic ligands in competition with [³H]-EPI was: TC-2429 (Kᵢ=2 nM)>A-85380=lobeline (Kᵢ=6 nM)>cytisine=methyllycaconitine=nicotine (Kᵢ=110-160 nM)>ABT-418=GTS-21═SIB-1508Y=carbachol (Kᵢ=0.5-2 μM)>dihydro-α-erythoidine=α-bungarotoxin=mecamylamine (Kᵢ>10 μM), a profile distinctly different from that of α4β2 and α7 receptors (as used herein, “TC” is an abbreviation for “Targacept Compound” (Targacept, Inc., Winston-Salem, N.C.)). Thus, α6β4β3α5 nicotinic receptors demonstrate unique pharmacology, and the results suggest contributions of all four subunits to receptor assembly.

### BRIEF DESCRIPTION OF THE FIGURES

FIG. 1 is a graph showing saturation [³H]-EPI binding to SH-EP1 α6β4β3α5 cell membranes. Saturation analysis was conducted using a concentration range of 0.01-2.0 nM of [³H]-EPI. Data are expressed as fmoles per mg of protein and represent one of three independent experiments, and the curves for total and specific binding were generated using nonlinear regression (a one-site model). Linear regression was used to plot nonspecific binding. The inset illustrates Scatchard transformation of the specific binding data.

FIG. 2 is a graph showing functional responses of SH-EP1 α6β34β3α5 cell evoked by acetylcholine (Ach) and carbachol (CAR). Data are expressed as a percent of control, and the curve was generated using nonlinear regression.

FIG. 3 is a graph showing competition of TC-8 for [³H]-EPI binding to SH-EP1 α6β4β2β3 cell membranes. Data are expressed as a fmol/mg protein, and the curve was generated using nonlinear regression (a one-site model).

FIG. 4 is a graph showing functional responses of SH-EP1 α6α4β2β3 and α4β2 cells evoked by cytosine (CYT). Data are expressed as a percent of control, and the curve was generated using nonlinear regression using sigmoidal dose-response equation with variable slope.

FIG. 5 is a graph showing functional responses of SH-EP1α6α4β2β3 and α4β2 cells evoked by nicotine (NIC). Data are expressed as a percent of control, and the curve was generated using nonlinear regression using sigmoidal dose-response equation with variable slope.

FIG. 6 is a graph showing inhibition of CAR-evoked responses in SH-EP1 α6α4β2β3 and α4β2 cells by pancuronium. Data are expressed as a percent of control (100 μM CAR), and the curve was generated using nonlinear regression using sigmoidal dose-response equation with variable slope.

FIG. 7 shows one example of a nucleotide (cDNA) sequence encoding a human nAChR α4 subunit. Sequences shown in FIGS. 7-12 include some vector-derived sequences upstream and downstream of the encoding sequences (encoding sequences shown as shaded). A sequence encoding the α4 subunit was blunt-ended and inserted at the EcRV site of pcDNA3.1zeo (conferring resistance to zeocin). See vector diagram at http://www.invitrogen.com/content/sfs/vectors/pcdna3.1 zeo_map.pdf.

FIG. 8 shows one example of a nucleotide (cDNA) sequence encoding a human nAChR β2 subunit. A sequence encoding the β2 subunit was blunt-ended and inserted into pcDNAhyg (conferring resistance to hygro) at EcoRV site. See vector diagram at: http://www.invitrogen.com/content/sfs/vectors/pcdna3.1hygro.pdf.

FIG. 9 shows one example of a nucleotide (cDNA) sequence encoding a human nAChR α6 subunit. A sequence encoding the α6 subunit was cloned as a XhoI fragment in pcDNA3.1+hygro (conferring resistance to hygromycin). See vector diagram at: http://www.invitrogen.com/content/sfs/vectors/pcdna3.1hygro.pdf. The sequence was also cloned as a KpnI (5′) and XbaI (3′) fragment in pcDNA3.1+ (conferring resistance to G418 (neomycin). See vector diagram at: http://www.invitrogen.com/content/sfs/vectors/pcdna3.1+.pdf.

FIG. 10 shows one example of a nucleotide (cDNA) sequence encoding a human nAChR β4 subunit. A sequence encoding the β4 subunit was cloned as an EcoRI (5′) and XhoI (3′) fragment in pcDNA3.1+zeo (conferring resistance to zeocin). See vector diagram at: http://www.invitrogen.com/content/sfs/vectors/pcdna3.1 zeo_map.pdf.

FIG. 11 shows one example of a nucleotide (cDNA) sequence encoding a human nAChR β3 subunit. A sequence encoding the β3 subunit was cloned as a HindIII(5″) and EcoRI (3′) fragment in pcDNA3.1 (+)neo (conferring resistance to G418 (neomycin)). See vector diagram at: http://www.invitrogen.com/content/sfs/vectors/pcdna3.1+.pdf. The sequence was also cloned as an EcoRV fragment in pEF6 myc/His A (conferring resistance to neomycin). See vector diagram at http://www.invitrogen.com/content/sfs/vectors/pef6mychis.pdf.

FIG. 12 shows one example of a nucleotide (cDNA) sequence encoding a human nAChR α5 subunit. A sequence encoding the α5 subunit was cloned as an EcoRV fragment in pEF6 myc/His A (conferring resistance to blasticydin). See vector diagram at http://www.invitrogen.com/content/sfs/vectors/pef6mychis.pdf.

### DESCRIPTION OF THE INVENTION

The present invention provides for the production of stably expressed α6*-nAChR. Although SH-EP1 cells were used in the Examples herein, nAChR subunit combinations have been stably transfected into other cell lines (Lukas, et al., 2002). Generally, cells that are null for expression of nAChR can be useful. SH-EP type cells can be obtained from the human neuroblastoma parental cell line SK—N—SH (See Ross, et al., 1983). SH-EP (“EP” for epithelial-like morphology) cells are morphologically distinguishable, lack expression of noradrenergic enzyme activity (tyrosine hydroxylase and dopamine-β-hydroxylase), and contain an isochromosome 1q (long arm of chromosome 1) (Ross, et al., 1983). Without wishing to be bound by any particular theory, it appears that such cells may be useful because they possess and properly express complex transmembrane proteins due to their neuronal lineage, while having lost expression of native nAChRs which could otherwise complicate analysis involving heterologous expression of nAChRs. (See Lukas, et al., 2002).

Other appropriate cells include HEK-293 (human embryonic kidney), IMR-32 human neuroblastoma cells, and CATH.a mouse neuronal cells. (See Lukas, et al. 2002, and citations therein). Those of skill in the art will recognize that the suitability of particular cell lines for heterologous expression of nAChRs according to the invention can be determined empirically, and that the foregoing guidance will provide direction for development of useful models beyond the examples expressly provided herein.

The present invention also provides methods using such cells for characterization of receptor binding and functional properties of the α6β3β4α5 and α6α4β2β3 subunit combinations. Radioligand competition studies determine the ability of a compound to bind to the receptor of interest and are thus the first step to identify relevant compounds. Functional studies assess the ability of the compound to cause a biological response. Ion efflux assays directly measure the ability of the compound to open the nAChR cation channel. Channel opening results in a number of downstream effects, including activation of second messenger systems, and ultimately, neurotransmitter release. Cells stably transfected with the α6β3β4α5 and α6α4β2β3 nAChR subunit combinations can be used to screen compounds in vitro for interaction with this subtype, leading to the identification of drugs that are effective in the treatment of diseases involving α6* nAChR. Compounds such as TC-2429, which show robust DA release, but little activity at α4β2 nAChR (Bencherif, et al., 1998) may instead be acting via α6-containing nAChR.

Parkinson's disease, Alzheimer's disease with Parkinsonism, Lewy Body dementia, and supranuclear palsy all involve the degeneration of DA neurons (Murray, et al., 1995; Rajput and Rajput, 2001; Martin-Ruiz, et al., 2002), a process responsible for the motoric deficits of these diseases. Several models of DA neuron degeneration have shown an association to α6* nAChR. When 6-OHDA is administered to rats to induce DA neuron cell death, there is a loss of α6 subunit mRNA (Charpantier, et al., 1998; Elliott, et al., 1998). Treatment with MPTP, a toxin specific for DA neurons, results in a significant loss of nicotine-evoked DA release in mice (Quik, et al., 2003). In the same animals, binding of ¹²⁵I-αCtxMII to α6* nAChR is significantly reduced and correlates to the loss of DA transporter protein, a marker for DA neurons (Quik, et al., 2003). A similar loss of ¹²⁵I-αCtxMII binding sites is seen in non-human primates treated with MPTP (Kulak, et al., 2002a; Kulak et al., 2002b). Agonists of α6* nAChR may be able to interact with residual α6* nAChR to increase DA release in patients with loss of DA neurons.

Dopaminergic and noradrenergic systems also play a role in substance abuse and attentional deficits. Nicotine agonists improve attention in rats, monkeys, and humans. In addition, compounds that block reuptake by dopamine and norepinephrine transporters have shown efficacy in alleviating attentional deficits in humans. The presence of α6* nAChR on these neurons indicates that this subtype may play a role in attention.

In one aspect of the present invention, cytisine, a partial agonist at α4β2, exhibited much greater efficacy at α6α4β2β3 than α4β2. Thus, the α6α4β2β3 cell line may be an appropriate screening tool for cytisine-like compounds for indications where such compounds may prove beneficial, such as for smoking cessation. Varenicline, a cytisine derivative and a partial α4β2 agonist, is currently in Phase III clinical trials for smoking cessation (Pfizer Inc., New York, N.Y.).

As used herein, “nAChR subunits” e.g., α4, α5, α6, β2, β3, β4, means polypeptides that comprise the human amino acid sequence as encoded by the corresponding nucleic acid sequences disclosed herein, or the structural and functional homologs of such polypeptides.

An “agonist” is a substance that stimulates its binding partner, typically a receptor. Stimulation is defined in the context of the particular assay, or may be apparent in the literature from a discussion herein that makes a comparison to a factor or substance that is accepted as an “agonist” or “partial agonist” of the particular binding partner by those of skill in the art. Stimulation may be defined with respect to an increase in a particular effect or function that is induced by interaction of the agonist or partial agonist with a binding partner and can include allosteric effects.

An “antagonist” is a substance that inhibits its binding partner, typically a receptor. Inhibition is defined in the context of the particular assay, or may be apparent in the literature from a discussion herein that makes a comparison to a factor or substance that is accepted as an “antagonist” of the particular binding partner by those of skill in the art. Inhibition may be defined with respect to an decrease in a particular effect or function that is induced by interaction of the agonist with a binding partner, and can include allosteric effects.

Accordingly, in one aspect, the invention relates to a nicotinic acetylcholinergic receptor comprising at least one each of the α6, β3, β4, and α5 subunits.

In another aspect, the invention relates to a nicotinic acetylcholinergic receptor comprising at least one each of the α6, α4, β2, and β3 subunits.

The present invention also relates to recombinant vectors comprising nucleic acid molecules encoding the nAChR subunits forming the receptors of the present invention, and to host cells containing the recombinant vectors. The invention also provides methods of making such vectors and host cells and for using them for production of receptors comprising the nAChR subunit polypeptides or peptides of the invention by recombinant techniques.

In another aspect, the invention relates to methods for identifying compounds that are agonists, antagonists, or partial agonists of human neuronal nicotinic acetylcholine receptors (nAChRs) comprising an α6 subunit. The method comprises contacting recombinant cells with a test compound, wherein the recombinant cells comprise nucleic acid encoding at least one human nAChR α6 subunit; the recombinant cells express an nAChR comprising at least one human α6 subunit encoded by the transfected nucleic acid; and the expressed nAChR comprises at least one nAChR α6 subunit. In other embodiments, the likelihood of agonist/antagonist/partial agonist functionality is initially evaluated by determining whether the test compound binds to the receptors expressed by the cells. In other embodiments, the functionality is evaluated by measuring ion flux, the electrophysiological response of the cells, whereby agonist, partial agonists, or antagonists of the nAChR are identified. In other embodiments, wherein test compounds are screened for agonism, the recombinant cells further comprise a DNA encoding a reporter gene operatively linked to DNA encoding a transcriptional control element wherein the activity of the transcriptional control element is regulated by the human neuronal nicotinic acetylcholine receptor. The reporter gene encodes a detectable gene product, wherein the detectable product is selected from the group consisting of mRNA and a polypeptide; and the interaction of the test compound with the nAChRs is measured by detecting the gene product encoded by the reporter gene. Antagonism can be similarly detected by exposing the test compound to the cells in the presence of a known agonist and measuring the reduction in the gene product encoded by the reporter gene. Alternatively, a reporter gene can be selected such that the nAChR agonist activity results in reduction of constitutive reporter gene activity, and agonist/antagonist activities are measured based on opposite effects on the reporter gene. In particular embodiments, the nAChR comprises subunits combinations selected from the group consisting of α6β3β4α5 and α6α4β2β3.

Guidance regarding selection of appropriate reporter genes can be found, for example, in Dunckley and Lukas (2003). One of skill in the art will recognize the reporter genes according to the present invention can be constructed using known techniques to couple transcriptional control regions of genes modulated by nicotinic receptors to constructs encoding standard reporter messages or expression products, e.g., as reviewed in Alam and Cook (1990). Reporter genes can include, but are not limited to, those encoding luciferase, β-galactosidase, xanthine-guanine phosphoribosyl transferase, and chloramphenicol acetyltransferase.

In another aspect, the invention provides methods of screening for compounds that are likely candidates for development as therapeutic or diagnostic agents relevant to disease states associated with functions mediated by α6-containing nAChRs. Compounds screened according to the methods of invention include those likely to be effective as therapeutic agents for treatment of diseases characterized by neuronal degeneration or malfunction, such as Parkinson's disease, Lewy Body dementia, supranuclear palsy, substance abuse, attentional deficits, retinal degeneration, and disorders of sensory integration. In particular embodiments, the likelihood of agonist/antagonist/partial agonist functionality is initially evaluated by determining whether test compounds bind to the receptors expressed by the cells. In other embodiments, the functionality is evaluated by measuring ion flux, the electrophysiological response of the cells, whereby agonist, antagonists, or partial agonists of the nAChR are identified. In particular embodiments, compounds relevant to treatment of such disorders are identified by evaluating their interaction and effects upon nAChRs comprising the subunit combination α6β3β4α5.

In another aspect, the invention provides methods of screening for compounds that are likely candidates for development of agents useful in the treatment of nicotine addiction, e.g. as an aid to smoking cessation. In particular embodiments, relevant compounds are identified by evaluating their interaction and effects upon nAChRs comprising the subunit combination α6α4β2β3.

Compounds that modulate the activity of nAChRs of the invention can also be evaluated by determining the effect of a test compound on the nAChR activity in cells (function or binding) by comparison to the effect on control cells that are substantially identical to the cells expressing a receptor of the invention but which do not express the receptors, or by comparison to the effect of the test compound on nAChR activity of the cells in the absence of the compound.

In yet another aspect, the invention comprises kits containing recombinant construction and instructions for the production of the nAChRs according to the invention. Such kits comprise a container or containers with expression vectors comprising nucleic acids encoding the subunit combinations of the nAChRs, and instructions from the expression of the same in appropriate cells in order to facilitate performance of the methods according to the invention.

In still another aspect, the invention relates to a process for making a compound that is an agonist, antagonist, or partial agonist of the nAChR of the invention, the process comprising carrying out one or more of the screening methods of the invention to identify a compound having the desired activity; and manufacturing the compound.

Nucleic Acids and Polypeptides

As is known in the art for any DNA sequence determined by an automated approach, any nucleotide sequence disclosed herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

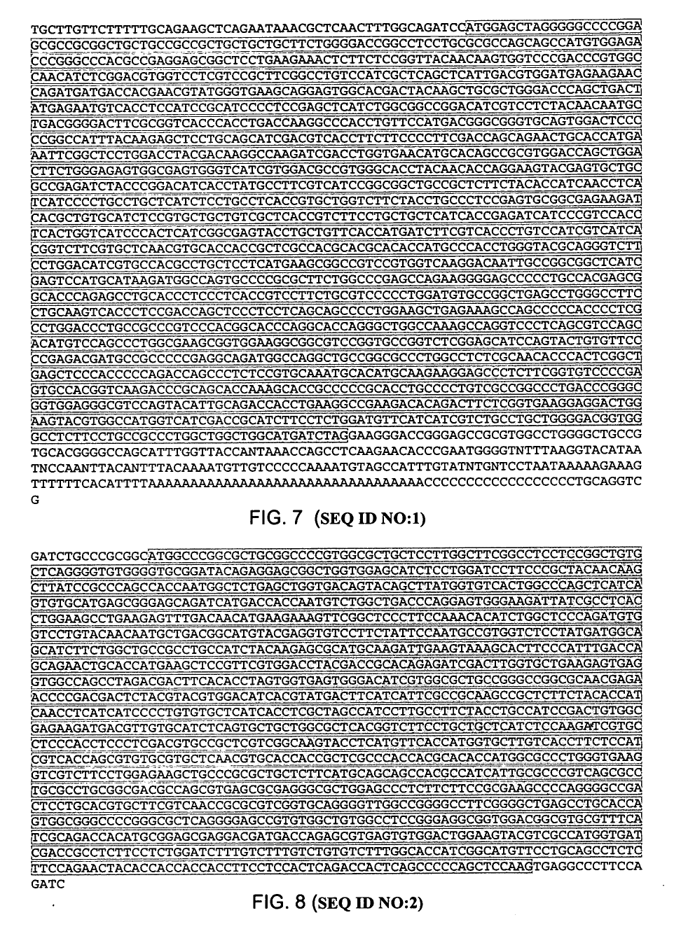

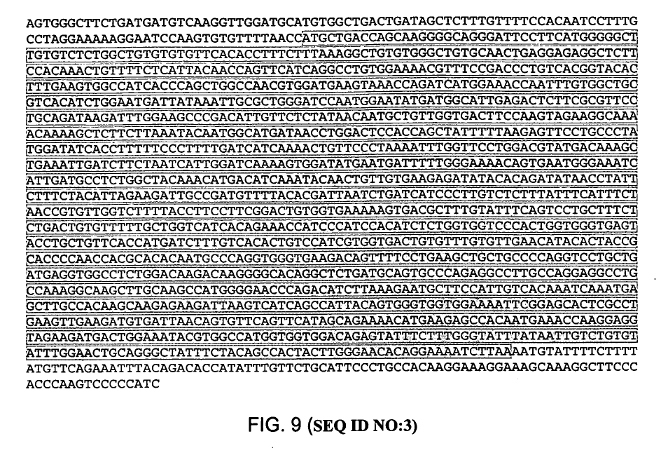

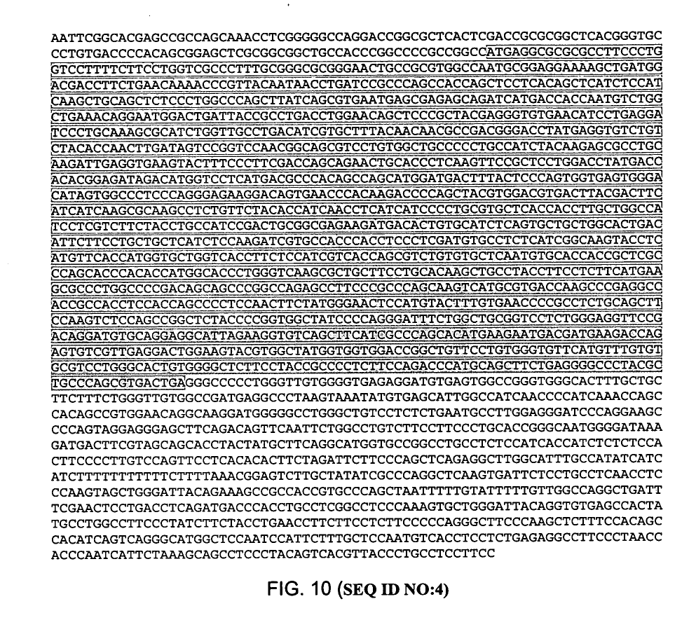

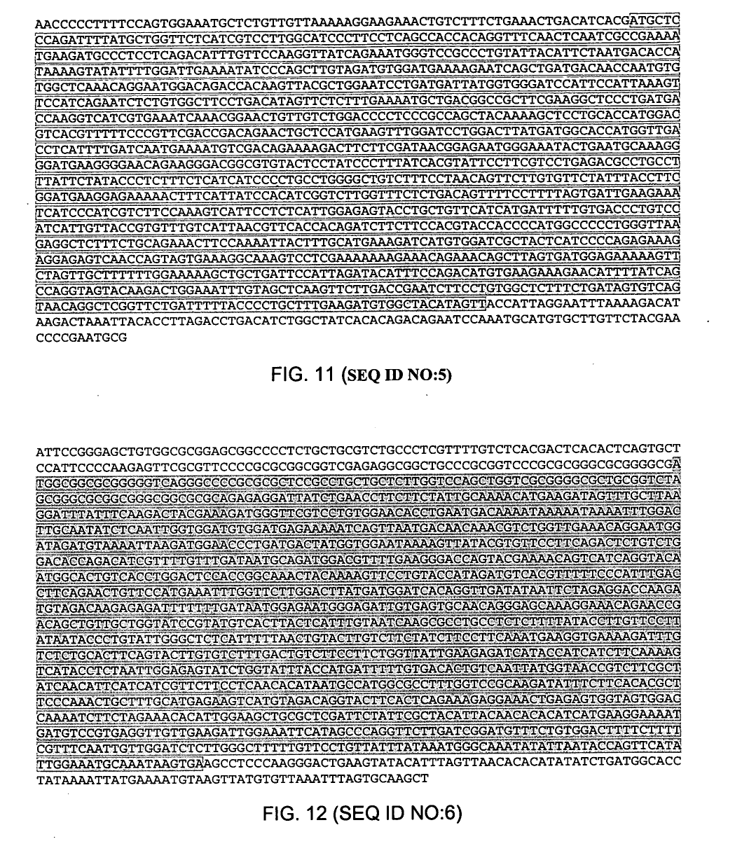

Using the information and guidance provided herein and knowledge in the art, such as the nucleotide sequences in FIGS. 7A, 8A, 9, 10, 11, and 12, a nucleic acid molecule of the present invention encoding an nAChR subunit polypeptide can be obtained using standard cloning and screening procedures, such as those for cloning cDNAs using mRNA as starting material.

As indicated, nucleic acid molecules of the present invention can be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention.

Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in FIGS. 7A, 8A, 9, 10, 11, and 12; DNA molecules comprising the coding sequence for the nAChR subunit proteins of the open reading frames shown in FIGS. 7A, 8A, 9, 10, 11, and 12; and DNA molecules which comprise a sequence substantially different from those described above but which, due to the degeneracy of the genetic code, still encode the nAChR subunit proteins. Of course, the genetic code is well known in the art. Thus, it would be routine for one skilled in the art to generate such degenerate variants. The present invention also includes other nucleic acid molecules and polypeptides defined according to the structural and functional requirements as disclosed herein.

Nucleic acids encoding portions of the nAChR subunits include nucleic acids determined by hybridization to those nucleic acids disclosed herein. Accordingly, the invention provides an isolated nucleic acid molecule comprising a polynucleotide which hybridizes under stringent hybridization conditions to a portion of the polynucleotide in a nucleic acid molecule of the invention described above, for instance, the polynucleotides disclosed in FIGS. 7A, 8A, 9, 10, 11, and 12. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

As indicated, nucleic acid molecules of the present invention that encode nAChR subunit polypeptides may include, but are not limited to, those encoding the amino acid sequences of the polypeptides, by themselves; the coding sequence for the polypeptides and additional sequences, such as those encoding a leader or secretory sequence, such as a pre-, or pro- or prepro-protein sequence; the coding sequence of the polypeptides, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example-ribosome binding and stability of mRNA; an additional coding sequence which codes for additional amino acids, such as those which provide additional functionalities.

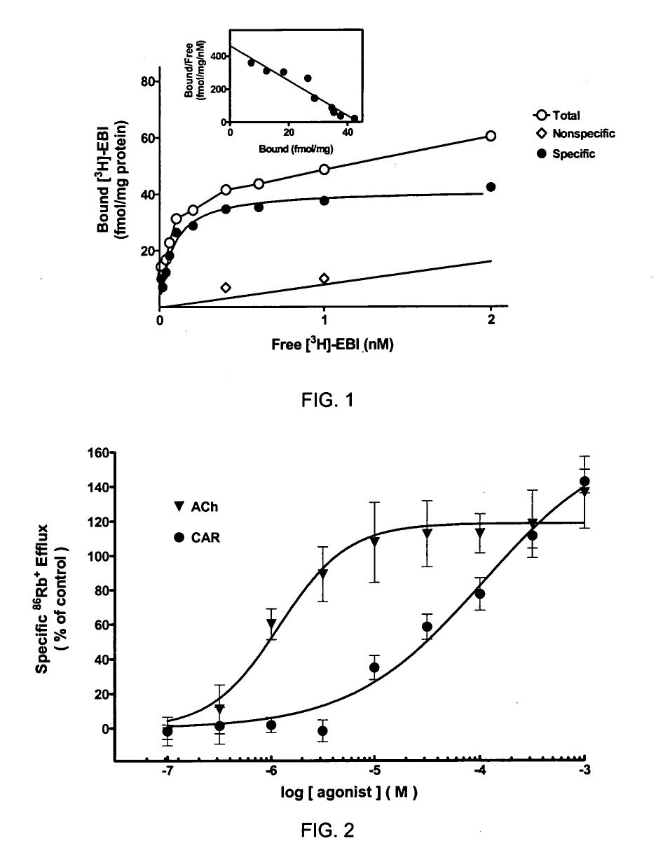

The present invention further relates to variants of the nucleic acid molecules of the present invention, which encode portions, analogs or derivatives of the nAChR subunit proteins. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques. The cDNA sequences disclosed herein (FIGS. 1-12, encoding sequences shaded (SEQ ID Nos: 1-6) are examples of nucleic acid molecules encoding the designated human nAChR subunits. Other sources for nucleotide sequences encoding polypeptides recognized as the designated subunits include references cited and incorporated herein, as well as those found in databases such as GENBANK.

Such variants include those produced by nucleotide substitutions, deletions or additions which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the nAChR subunit proteins or portions thereof. Also especially preferred in this regard are conservative substitutions.

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding the nAChR subunit polypeptides having the complete amino acid sequence encoded by the nucleic acid sequences shown in FIGS. 7A, 8A, 9, 10, 11, or 12 or any other sequence defined according to the present invention; (b) a nucleotide sequence encoding nAChR subunit polypeptides, but lacking the N-terminal methionine; and (c) a nucleotide sequence complementary to any of the nucleotide sequences in (a) or (b).

By a polynucleotide having a nucleotide sequence at least, for example, 95% “identical” to a reference nucleotide sequence encoding an nAChR subunit polypeptide is intended that the nucleotide sequence of the polynucleotide is identical to the reference sequence except that the polynucleotide sequence may include up to five point mutations per each 100 nucleotides of the reference nucleotide sequence encoding the nAChR subunit polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIGS. 7A, 8A, 9, 10, 11, or 12 can be determined conventionally using, known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleofide sequence may include up to five point mutations per each 100 nucleotides of the reference nucleotide sequence encoding the nAChR subunit polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in FIGS. 7A, 8A, 9, 10, 11, or 12, the ORF (open reading frame), or any fragment specified as described herein, e.g. domains of the nAChR subunit.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a match/alignment of the first 10 bases at the 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the nucleic acid sequence shown in FIGS. 7A, 8A, 9, 10, 11, and 12 will encode a polypeptides “having nAChR subunit protein activity.” In fact, because degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having nAChR subunit protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U., et al., “Deciphering the Message in Protein Sequences: Tolerance to Amino Acid Substitutions,” Science 247:1306-1310 (1990), wherein the authors indicate that proteins are surprisingly tolerant of amino acid substitutions. It will be recognized in the art that some amino acid sequences of the nAChR subunit polypeptides can be varied without significant effect of the structure or function of the protein. If such differences in sequence are contemplated, it should be remembered that there will be critical areas on the protein which determine activity.

Thus, the invention further includes variations of the nAChR subunit polypeptides which show substantial nAChR subunit polypeptide activity or which include substantially all functional regions of nAChR subunit protein. Such mutants include deletions, insertions, inversions, repeats, and type substitutions. As indicated above, guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptides encoded by the nucleotides of FIGS. 7A, 8A, 9, 10, 11, or 12, or any other sequence defined according to the present invention, may be (i) one in which one or more of the amino acid residues (e.g., 3, 5, 8, 10, 15 or 20) are substituted with a conserved or non-conserved amino acid residue (preferably a conserved amino acid residue) and such substituted amino acid residue may or may not be one encoded by the genetic code, or (ii) one in which one or more of the amino acid residues includes a substituent group (e.g., 3, 5, 8, 10, 15 or 20), or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

As indicated, changes are preferably of a minor nature, such as conservative amino acid substitutions that do not significantly affect the folding or activity of the protein:

Conservative Amino Acid Substitutions

| | |
|---|---|
| Aromatic: | Phenylalanine, Tryptophan, Tyrosine |
| Hydrophobic: | Leucine, Isoleucine, Valine |
| Polar: | Glutamine, Asparagine |
| Basic: | Arginine, Lysine, Histidine |
| Acidic: | Aspartic Acid, Glutamic Acid |
| Small: | Alanine, Serine, Threonine, Methionine, Glycine |

Of course, the number of amino acid substitutions a skilled artisan would make depends on many factors, including those described above. Generally speaking, the number of substitutions for any given subunit polypeptide will not be more than 50, 40, 30, 25, 20, 15, 10, 5 or 3.

Amino acids in the nAChR subunit proteins of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith, et al., J. Mol. Biol. 224:399-904 (1992) and de Vos, et al. Science 255:306-312 (1992)).

Accordingly, the present invention further provides polypeptides having one or more residues deleted from the amino and/or carboxy terminus of the amino acid sequence of the nAChR subunit polypeptide encoded by the nucleic acid sequences shown in FIGS. 7A, 8A, 9, 10, 11, or 12, or any other sequence defined according to the present invention.

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host. For example, recombinantly produced versions of the nAChR subunit polypeptides can be substantially purified by the one-step method described in Smith and Johnson, Gene 67:31-40 (1988).

By a polypeptide having an amino acid sequence at least, for example, 95% “identical” to a reference amino acid sequence of an nAChR subunit polypeptide is intended that the amino acid sequence of the polypeptide is identical to the reference sequence except that the polypeptide sequence may include up to five amino acid alterations per each 100 amino acids of the reference amino acid of the nAChR subunit polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence encoded by the nucleic acid sequence shown in FIGS. 7A, 8A, 9, 10, 11, or 12, or any other sequence defined according to the present invention, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in FIGS. 7A, 8A, 9, 10, 11, or 12, or any other sequence defined according to the present invention, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty-1, Joining Penalty=20, Randomization Group Length=0, Cutoff Score=1, Window Size=sequence length, Gap Penalty-5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues of the query (reference) sequence that extend past the N- or C-termini of the subject sequence are considered for the purposes of manually adjusting the percent identity score. That is, only residues which are not matched/aligned with the N- or C-termini of the query sequence are counted when manually adjusting the percent identity score.

For example, a 90 amino acid residue subject sequence is aligned with a 100 residue query sequence to determine percent identity. The deletion occurs at the N-terminus of the subject sequence and therefore, the FASTDB alignment does not show a match/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The invention encompasses nAChR subunit polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition of an N-terminal methionine residue as a result of prokaryotic host cell expression. The polypeptides may also be modified with a detectable label, such as an enzymatic, fluorescent, isotopic or affinity label to allow for detection and isolation of the protein.

### EXAMPLES

Animals. Female Sprague-Dawley rats weighing 150-200 g (Charles River Laboratories, Raleigh, N.C.) were housed one per cage (12/12-hr light/dark cycle) with free access to food and water. Experimental protocols involving the animals were in accordance with the Declaration of Helsinki and the National Institutes of Health Guide for the Care and Use of Laboratory Animals, and were approved by the Institutional Animal Care and Use Committee at the Targacept, Inc.

cDNA preparation. All cDNAs containing inserts corresponding to human nAChR subunit coding regions were prepared according to the MAXiprep (Marligen Biosciences, Inc.) protocol. The indicated human nAChR subunit subcloned into the indicated expression vector was used for transfection either as circular DNA or as linearized plasmid cut with the indicated restriction endonuclease: α4, in pcDNA3.1−hygro (conferring hygromycin resistance), uncut; α5, in pEF6 (conferring blasticydin resistance), cut with Fsp I; α6, in pcDNA3.1−hygro, cut with Fsp Í; α6, in pcDNA3.1−neo (conferring resistance to G418 (neomycin)), cut with Pvu I; β2, in pcDNA3.1−zeo (conferring resistance to zeocin), cut with Pvu I; β2 in pcDNA3.1−zeo, uncut; β3, in pcDNA3.1−neo, cut with Pvu I; β3, in pEF6, cut with Fsp I; β4, in pcDNA3.1 zeo, cut with Pvu I. Final constructs were verified by restriction mapping and complete sequencing of the insert

Preparation of Cell Lines. Sh-EP1 Human Epithelial Cell Line (Provided by Dr. June Biedler, Sloan Kettering Institute for Cancer Research) were grown in DMEM supplemented with 10% horse serum, 100 U/ml penicillin, 100 μg/ml streptomycin and 0.25 μg/ml amphotericin B (all from Life Technologies, Inc., Gaithersberg, Md.) plus 5% fetal bovine serum (Hyclone, Logan, Utah) in a humidified atmosphere containing 5% CO₂ in air at 37° C. Cells were transfected using either the SUPERFECT technique (Qiagen) (α6α4β2β3) or an electroporation procedure (α6β3β4α5), and no notable differences were observed in transfection efficiency for any of the SH-EP1 cell derivatives. Generally, for the SUPERFECT technique, 10 μg of DNA dissolved in TE buffer, pH 7.4 (minimum DNA concentration of 0.1 μg/μl) was diluted in serum-, protein-, and antibiotic-free DMEM to a total volume of 300 μl before adding 60 μl of SUPERFECT Transfection Reagent. The sample was mixed and incubated 10-15 min at room temperature (22±1° C.) before addition of 3 ml of complete (serum-supplemented DMEM containing penicillin-streptomycin-amphotericin B). The mixed sample was then added to one 100-mm plate containing ˜0.8-1.6 million cells (40-80% confluence) that had been previously rinsed once with 10 ml of warm phosphate-buffered saline (PBS). The cells were transferred to an incubator for 2-3 hr of maintenance at 37° C. in 5% CO₂ in air. Transfection medium was then aspirated, and cells were rinsed 34× with 10 ml of warm PBS before addition of fresh, complete DMEM and maintenance at 37° C. in 5% CO₂ in air for another 24 hr. Medium was then supplemented with the selection antibiotic. In cases where electroporation (BIORAD GENE PULSAR model 1652076 with pulse control module model 1652098 operating at 960 μF and 0.20 kV/cm (t=28-36 ms)) was used, ˜2 million cells from one confluent 100-mm plate were harvested mechanically under a stream of fresh medium after brief exposure to 2 ml of trypsin solution. The cell suspension was transferred to a 15-ml conical (sterile) tube and centrifuged at 7000 rpm for 4 min to pellet the cells. After removal of medium by aspiration, cells were resuspended in 800-μl of HEBS buffer (20 mM HEPES, 87 mM NaCl, 5 mM KCl, 0.7 mM NaHPO₄, 6 mM dextrose, pH 7.05). After addition of 100 μg DNA suspended in TE, the sample was triturated to ensure a uniform suspension and transferred to a sterile electroporation cuvette. After electroporation, the sample was allowed to settle for 10-15 min before being added to 10-ml of fresh, complete DMEM, mixed, and transferred to a 100-mm plate. The transfected cells were then incubated for 24 hr at 37° C. in 5% CO₂ in air before medium was supplemented with the selection antibiotic. Regardless of the method used for transfection, cell growth was monitored until ring cloning or the “stab-and-grab” technique was used to isolate single, transfected cell colonies, which were then expanded. RT-PCR was conducted to verify the presence of all transfected nAChR subunit mRNA. The cell line SH-EP1-α6β4β3α5 was created by sequential transfection with pcDNA3.1−hygro-hα6, pcDNA3.1−zeo-hβ4, pcDNA3.1−neo-hβ3, and pEF6-hα5. A SH-EP1-hα4β2 cell line already established (transfected with pcDNA3.1−hygro-hα4 and pcDNA3.1−zeo-hβ2) was transfected again with pcDNA3.1−neo-hα6 and then with pEF6-hα5 to create the SH-EP1-hα6α4β2β3 cell line.

Cell culture. Cells were grown in DMEM (high glucose, bicarbonate-buffered, with 1 mM sodium pyruvate and 8 mM L-glutamine) supplemented with 10% horse-serum, 100 U/ml penicillin, 100 μg/ml streptomycin and 0.25 μg/ml amphotericin B (all from Life Technologies, Inc., Gaithersberg, Md.) plus 5% fetal bovine serum (Hyclone, Logan, Utah) on 100-mm diameter plates in a humidified atmosphere containing 5% CO₂ in air at 37° C. (Lukas, 1986; Lukas, et al., 1993). 130 μg/ml Hygromycin B (CalBioChem), 400 μg/ml G418 (CalBioChem), 250 μg/ml Zeocin, and 5 μg/ml Blasticidin (Invitrogen) were included in the medium to select for transfected cells.

RNA preparation and Reverse transcription polymerase chain reaction (RT-PCR). Total cRNA was isolated from cells growing at approximately 80% confluency in a 100-mm culture dish using 2 ml of Trizol reagent (Bethesda Research Laboratories, Gaithersburg, Md.). Prior to RT-PCR, RNA preparations were treated with Dnase I (Ambion, Austin, Tex.) to remove residue genomic DNA contamination. Typically, 40 μg of RNA was incubated with 4 units of Dnase I in a 50-μl reaction at 37° C. room temperature for 30 min, and then the DNase I was inactivated by addition of 5 μl of 25 mM EDTA and incubation at 65° C. for 10 min. RT was carried out using 2 μg of DNA-free total RNA, oligo d(T)12-18 primer, and a Superscript II preamplification system (BRL) in a 20-μl reaction. At the end of the RT reaction, reverse transcriptase was deactivated by incubation at 75° C. for 10 min, and RNAs were removed by adding 1 unit of RNaseH followed by incubation at 37° C. for 30 min. Reaction excluding reverse transcriptase was also conducted as RT negative control. PCR was performed using 1 μl of cDNA preparation, 1 μl of 10 μM each of 5′ and 3′ gene-specific primers, 1 μl of 10 mM dNTP, and 2.5 units of REDTAQ (Sigma, St. Louis, Mo.) in a 50 μl of reaction volume. Amplification reactions were carried out in a RoboCycler (Stratagene; La Jolla, Calif.) for 35 amplification cycles at 95° C. for 1 min, 55° C. for 90 seconds, and 72° C. for 90 sec, followed by an additional 4-min extension at 72° C. One-tenth of each RT-PCR product was then resolved on a 1% agarose gel, and sizes or products were determined based on migration relative to mass markers loaded adjacently.

Preparation of Cell Membranes for Receptor Binding. Cells were mechanically scraped, harvested in ice-cold Dulbecco's phosphate-buffered saline (PBS, # 21300, Invitrogen Corporation, Carlsbad, Calif.), pH 7.4, then homogenized with a polytron (Brikmann Instruments, Westbury, N.Y.) at setting 6 for 15 sec. Combined homogenate (18 mL) was centrifuged at 40,000×g for 20 min (4° C.). The pellet was resuspended in 12 mL of ice-cold PBS and centrifuged again. The final pellet was resuspended in 10 mL of PBS. Resulted membrane preparation contained 1.2-1.5 mg/mL of total protein, as determined using the Bradford dye-binding method (Bradford, 1976) with bovine serum albumin as the standard.

[³H]-Epibatidine Binding. For the saturation binding assay, 0.1 to 2.0 nM [³H]-EPI (final concentrations; PerkinElmer Life Sciences, 56.2 Ci/mmol) was used to probe α6-comprising binding sites on the cell membranes. Competition binding assay was performed using 0.5 nM [³H]-EPI (final concentration).

Test samples were assayed in PBS (0.9 mM CaCl₂; 2.67 mM KCl; 1.47 mM KH₂PO₄; 0.49 mM MgCl₂; 137.93 mM NaCl and 4.29 mM Na₂HPO₄, pH 7.4), for a final volume of 200 μl in either 48-well or 96-well plates. Each well contained 50 μL of test compound at the desired concentration, 50 μL of 4×[³H]-EPI stock solution and 100 μL of membrane suspension and was performed in triplicate, at minimum. Samples were incubated for 2 hr at room temperature with gentle agitation. Samples defining total binding included buffer instead of test compound. Nonspecific binding was measured in the presence of 100 μM nicotine.

For 48-well plates, binding was terminated by immediate filtration onto GF/B filters (presoaked in 0.3% PEI) using a 48-sample, semi-auto harvester (Brandel, Gaithersburg, Md.), followed by washing 3 times with ice-cold buffer. Filters were transferred into scintillation vials filled with 3 mL of cocktail. Radioactivity was measured after 8-12 hr using a liquid scintillation analyzer (model Tri-Carb 2200CA, PerkinElmer Life Sciences Inc., Boston, Mass.). Data were expressed as disintegrations per minute (DPMs) and were converted to an absolute amount (fmoles) of bound [³H]-EPI per mg of protein, or as a percent of control [³H]-EPI binding (total—nonspecific).

For 96-well plates, incubation was terminated by dilution with ice-cold PBS and immediate filtration onto GF/B filter plate (presoaked in 0.3% PEI) using a 96-sample, semi-auto harvester (Brandel, Gaithersburg, Md.). After washing 3 times with ˜350 μl of ice-cold buffer, the filter plate was dried for 60 min in an oven at 49° C., bottom-sealed and each well filled with 40 μl of cocktail. After 60 min, the filter plate was top-sealed and radioactivity measured using a Wallac 1450 Microbeta liquid scintillation counter. Data were expressed as counts per minute (CPMs) and were converted to percent of control [³H]-EPI binding (total—nonspecific).

IC₅₀ value, the concentration of drug that inhibits specific binding by 50%, was determined by a nonlinear regression, fitting data from the competition binding assay to a one-site model. The inhibition constant (Kᵢ) for each drug was calculated from IC₅₀ values using the Cheng-Prusoff equation [Kᵢ=IC₅₀/(1+ligand/K_{d})]. Pseudo Hill slope (nH) was determined by fitting data to a sigmoidal dose-response equation (variable slope): % binding=Bottom+(Top−Bottom)/[1+10^{(logIC50-X)·n}], where X is the logarithm of inhibitor concentration and n is the slope.

⁸⁶Rb⁺-flux Assay. Cells were harvested at confluence from 100-mm plates by mild trypsinization (Irvine Scientific, Santa Ana, Calif.) before being resuspended in complete medium and evenly seeded at a density of one confluent 100-mm plate per 24-well plate (Falcon; ˜100-125 μg of total cell protein per well in a 500 μl volume). After cells had adhered (generally overnight, but no sooner than 4 hr later), medium was removed and replaced with 250 μl per well of complete medium supplemented with ˜300,000 cpm of ⁸⁶Rb⁺ (NEN; counted at 40% efficiency using Cerenkov counting and the Packard TriCarb 1900 Liquid Scintillation Analyzer).

After at least 4 hr and typically overnight, ⁸⁶Rb⁺ efflux was measured using the “flip-plate” technique (Lukas et al., 2002). Briefly, after aspiration of the bulk of ⁸⁶Rb⁺ loading medium from each well of the “cell plate,” each well containing cells was rinsed three times with 2 ml of fresh ⁸⁶ Rb⁺ efflux buffer (130 mM NaCl, 5.4 mM KCl, 2 mM CaCl₂, 5 mM glucose, 50 mM HEPES, pH 7.4) to remove extracellular ⁸⁶Rb⁺. Following removal of residual rinse buffer by aspiration, the flip-plate technique was used again to simultaneously introduce fresh efflux buffer containing drugs of choice at indicated final concentrations from a 24-well “efflux/drug plate” into the wells of the cell plate. After a 3-min incubation, the solution was “flipped” back into the efflux/drug plate, any remaining medium was removed by aspiration, and the remaining and cells in the cell plate were lysed and suspended by addition of 2 ml of 0.1M NaOH, 0.1% sodium dodecyl sulfate to each well. Suspensions in each well were then subjected to Cerenkov counting (Wallac Micobeta Trilux 1450; 25% efficiency) after placement of inserts (Wallac 1450-109) into each well to minimize cross-talk between wells.

For each experiment, normalization and quality control measurements were made of total ⁸⁶Rb⁺ efflux in samples containing a fully efficacious dose of 1 mM carbamylcholine and of non-specific ⁸⁶Rb⁺ efflux measured using either samples containing 1 mM carbamylcholine plus 100 μM mecamylamine, which gave full block of agonist-induced or spontaneous, nAChR-mediated ion flux, or samples containing efflux buffer alone to assess any contributions due to spontaneous nAChR-mediated ion flux. Intrinsic agonist activity of test drugs was ascertained in samples containing that drug only at different concentrations and was normalized, after subtraction of non-specific efflux, to specific efflux assessed using carbamylcholine and efflux-buffer-only controls. Antagonist activity was determined for test drugs at different concentrations in the presence of 1 mM carbamylcholine and was normalized, after subtraction of non-specific efflux, to specific efflux ascertained using carbamylcholine and efflux buffer-only controls. ⁸⁶Rb⁺ in both cell plates and efflux/drug plates was periodically determined to ensure material balance (i.e., that the sum of ⁸⁶Rb⁺ released into the efflux/drug plate and ⁸⁶Rb⁺ remaining in the cell plate were the same for each well) and to determine efficiency of ⁸⁶Rb⁺ loading (the percentage of applied ⁸⁶Rb⁺ actually loaded into cells). Specific ⁸⁶Rb⁺ efflux was determined in absolute terms and as a percentage of loaded ⁸⁶Rb⁺. Depending on cell density and the concentration of ⁸⁶Rb⁺ in the loading medium, SH-EP1-hα4β2 cells typically display specific efflux of 5,000-15,000 cpm per sample of ⁸⁶Rb⁺ with a ratio of total to non-specific efflux of 10:1 and with total efflux being about one-half of loaded ⁸⁶Rb⁺.

Data analysis Parameters [dissociation constant K_{D} and maximum binding level Bₘₐₓ; B=Bmax/(1+(K_{D}/X)ⁿ)] for specific radioligand binding were determined from nonlinear graphic analysis (Prism software, GraphPad, San Diego, Calif.) of plots of specific binding, B, as a function of the free concentration of radioligand, X, and for Hill coefficient, n, for each sample, where specific binding was defined as total minus non-specific binding, and non-specific binding was calculated from linear regression analysis of H-EBDN binding in the presence of 100 μM nicotine. A Scatchard analysis was also done for illustrative purposes, but not to determine specific binding parameters. Specific binding, B, as a function of competing drug concentration, X, was plotted and fit to the Hill equation, B=Bₘₐₓ/(1+(X/IC₅₀)ⁿ) for competing drug concentration to give half-maximal inhibition of radioligand binding, IC₅₀, control specific binding Bₘₐₓ, and the Hill slope, n (Prism).

Ion flux assays were also fit to the Hill equation but made measures of specific ion flux, F, as a percentage of control, Fₘₐₓ, for EC₅₀ (n>0 for agonists) or IC₅₀ (n<0 for antagonists; Prism). In some cases, biphasic dose-ion flux response curves were evident and were fit to a two-phase Hill equation from which EC₅₀ and Hill coefficients for the rising, agonist phase, and IC₅₀ and Hill coefficients for the falling, self-inhibitory phase could be determined (Prism). Most ion flux data was fit allowing maximum and minimum ion flux values to be determined by curve fitting, but in some cases where antagonists or agonists had weak functional potency, minimum ion flux was set at 0% of control or maximum ion flux was set at 100% of control, respectively.

Materials. [³H]-Epibatidine ([³H]-EPI, 56.2 Ci/mmol) and [³H]-S-(−)-nicotine ([³H]-NIC, 81.5 Ci/mmol) and ⁸⁶RbCl were purchased from PerkinElmer Life Science (Boston, Mass.). [³H]-methyllycaconitine ([³H]-MLA, 25.4 Ci/mmol) and cold MLA and NUD were purchased from Tocris Cookson Ltd. (Briston, UK). CYT, A-85380, S-(−)-NIC, LOB, DHβE; CAR, MCC, EPI, α-Btx; α-D-glucose, polyethylenimine (PEI) and bovine serum albumin were purchased from Sigma-Aldrich (St. Louis, Mo.). Remaining chemicals in the binding and release assay buffers were purchased from Fisher Scientific (Pittsburgh, Pa.).

Compound Abbreviations. ABT-418, (S)-3-methyl-5-(1-methyl-2-pyrrolidinyl)isoxazole; A-85380, 3-[2(S)-azetidinylmethoxy]pyridine; α-Btx, α-bungarotoxin; CAR, carbachol; CYT, cytosine; DHβE, dihydro-β-erythoidine; EPI, epibatidine; DMPP, 1,1-dimethyl-4-phenylpiperazinium; GTS-21, (2.4)-dimethoxybenzylidene anabaseine; LOB, lobeline; MEC, mecamylamine; MLA, methyllycaconitine; MCC, methylcarbamylcholine; MPTP, 1-methyl-4-phenyl-1,2,3,6-tetrahydropyridine; NIC, nicotine; NUD, nudikauline; SIB-1508Y, altinicline.

Reverse transcription-polymerase chain reaction (RT-PCR) analysis was used to identify the nAChR subunits that were stably expressed in SH-EP1 cells. Monoclones expressing mRNA for all four subunits (α6, β3, β4 and α5; or α6, α4, β2, and β3) were selected for further study. [³H]-epibatidine (EPI) binding was conducted to provide additional evidence for nAChR expression.

### Example 1 α6β3β4α5

Radioligand binding studies were conducted on membranes from SH-EP1 cells expressing α6β3β4α5 nAChR. Specific, saturable [³H]-EPI binding was observed (FIG. 1) with K_{D}=70 μM and Bₘₐₓ=41 fmol/mg. Data was best fit to a one-site model (R²=0.97; F_{2,6}=0.539: p=0.61). See Table 1 below. Standard nicotinic ligands were used to characterize the binding profile of α6β3β4α5 nAChR. The rank order of binding potency for nicotinic ligands in competition with [³H]-EPI was: TC-2429 (Kᵢ=2 nM)>lobeline=A-85380 (Ki=7-9 nM)>cytisine=methyllycaconitine=nicotine (Kᵢ=150-165 nM)>ABT-418=SIB-1508Y=methylcarbachol=GTS-21=carbachol (Kᵢ=0.9-3.5 μM)>dihydro-β-erythoidine=α-bungarotoxin=mecamylamine (Kᵢ>10 μM). This profile was distinctly different from that of α4β2 and α7 receptors (Table 1).

TABLE 1
Binding profile (Ki values) of nAChR ligands.

| | α6β3β4α5 | α4β2 | α7 |
|---|---|---|---|
| | [³H]-EPI Binding | [³H]-NIC Binding | [³H]-MLA Binding |
| | SH-EP1 cells | Rat Brain | Rat brain |
| Compound | Ki (nM) | | |
| EPI | 0.07 | | |
| TC-2429 | 2 | 1 | 50 |
| (-)-LOB | 7 | 15 | >10000 |
| A-85380 | 9 | 3 | 1200 |
| (-)-CYT | 150 | 1 | 1160 |
| (-)-NIC | 160 | 3 | 370 |
| MLA | 160 | | 2 |
| NUD | 370 | | |
| ABT-418 | 850 | 39 | |
| SIB-1508Y | 950 | 200 | |
| MCC | 970 | | |
| GTS-21 | 1400 | 20 (Briggs et al, | 2000 (Briggs et al, |
| | | 1997) | 1997) |
| CAR | 3500 | | |
| DHβE | >10,000 | 26.4 | 5000 |
| α-Btx | >10,000 | >10000 | |

In addition, selected TC compounds were used for comparison of their potencies between α6β3β4α5, α4β2 and α7 nAChRs. Distinct chemical families of TC compounds revealed different binding patterns at the three nAChR subtypes (Table 2).

TABLE 2
Binding profile (Ki values) of nAChR ligands.

| | α6β3β4α5 | α4β2 | α7 |
|---|---|---|---|
| | [³H]-EPI Binding | [³H]-NIC Binding | [³H]-MLA Binding |
| | SH-EP1 cells | Rat Cortex | Rat Hippocampus |
| Compound | Ki (nM) | | |
| TC-1 | 3 | 1-3 | 930 |
| TC-2 | 5 | 1 | 15 |
| TC-3 | 23 | 0.03 | 61 |
| TC-4 | 63 | 7 | 196 |
| TC-5 | 651 | 46 | 51 |
| TC-6 | 1540 | 3080 | 15 |
| TC-7 | 1753 | 10 | >10,000 |
| TC-8 | 2200 | 15-22 | >10,000 |
| TC-9 | >10,000 | 4-9 | >10,000 |
| TC-10 | >10,000 | 17-34 | 2000 |
| TC-11 | >10,000 | 32-70 | >10,000 |

Function of α6β3β4α5 subunit combinations was assessed using ⁸⁶Rb⁺ efflux assays. Both ACh and CAR activate ⁸⁶Rb⁺ efflux responses from SH-EP1 cells expressing α6β3β4α5-nAChR (FIG. 2). However, there was no ion flux responses in the absence of α6 subunits, when β3, β4, or α5 subunits are expressed alone or in any combination. Thus, α6 subunit inclusion in α6β3β4α5 nAChR is both necessary and sufficient for formation of functional nAChR.

### Example 2 α6α4β2β3

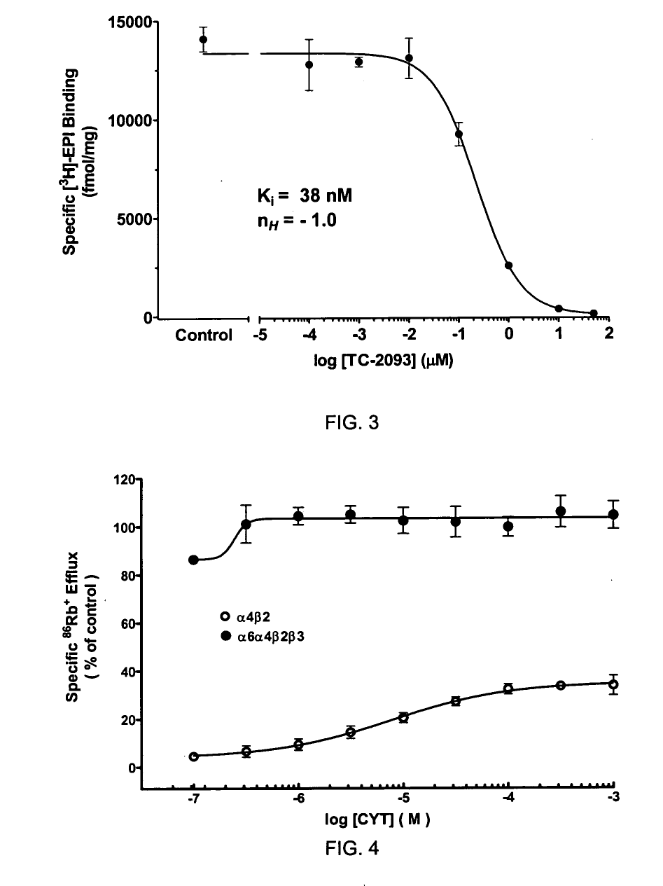

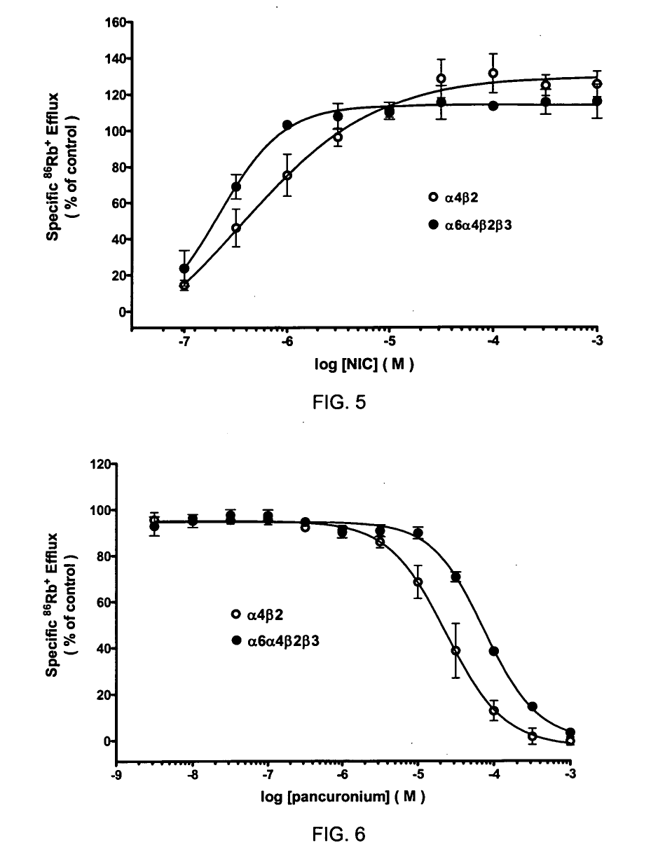

High levels of [³H]-EPI binding to α6α4β2β3 nAChRs in SH-EP1 cells was observed and can be displaced with TC-8 (Kᵢ=38 nM, FIG. 3). In functional studies, CYT had higher efficacy and agonist potency at α6α4β2β3 nAChR than at α4β2-nAChR (FIG. 4). NIC also exhibited higher functional agonist potency at α6α4β2β3-nAChR than at α4β2-nAChR (FIG. 5). These findings indicate that inclusion of α6 subunits in assemblies that also contain α4 subunits alters functional pharmacological properties. This interpretation is supported by lower sensitivity of α6α4β2β3-nAChR to functional blockade by pancuronium than for α4β2-nAChR (FIG. 6). Tandem immunoprecipitation-western analyses indicates that α6 and α4 subunits are indeed co-assembled in expressed α6α4β2β3-nAChR.

### REFERENCES

Alam, J. and J. L. Cook, “Reporter genes: application to the study of mammalian gene transcription,” Anal. Biochem., 188(2): 245-54 (1990).
Azam, L., et al., “Expression of neuronal nicotinic acetylcholine receptor subunit mRNAs within midbrain dopamine neurons,” J. Comp. Neurol., 444: 260-74 (2002).
Barabino, B., et al., “An alpha4beta4 nicotinic receptor subtype is present in chick retina: identification, characterization and pharmacological comparison with the transfected alpha4beta4 and alpha6beta4 subtypes,” Mol. Pharmaco., 59: 1410-17 (2001).
Bencherif, M., et al., “The heterocyclic substituted pyridine derivative (+/−)-2-(−3-pyridinyl)-1-azabicyclo[2.2.2]octane (RJR-2429): a selective ligand at nicotinic acetylcholine receptors,” J. Pharmacol. Exp. Ther., 284: 886-894 (1998).
Bencherif, M. and J. D. Schmitt, “Targeting neuronal nicotinic receptors: a path to new therapies,” Current Drug Targets—CNS & Neurological Disorders, 1: 349-357 (2002).
Blanchfield, J. T., et al., “Synthesis, structure elucidation, in vitro biological activity, toxicity, and Caco-2 cell permeability of lipophilic analogues of alpha-conotoxin MII,” J. Med. Chem., 46(7): 1266-72 (2003).
Boorman, J. P., et al., “Stoichiometry of human recombinant neuronal nicotinic receptors containing the b3 subunit expressed in Xenopus oocytes,” J. Phys., 529: 565-577 (2000).
Boulter, J., et al., “Functional expression of two neuronal nicotinic acetylcholine receptors from cDNA clones identifies a gene family,” Proc. Natl. Acad. Sci. USA, 84: 7763-7767 (1987).
Boulter, J., et al., “Isolation of a cDNA clone coding for a possible neural nicotinic acetylcholine receptor alpha-subunit,” Nature (Lond), 319: 368-374 (1986).
Bradford, M. M., “A rapid and sensitive method for the quantitation of microgram quantities of protein utilizing the principle of protein-dye binding,” Anal. Biochem., 72: 248-54 (1976).
Cartier, G. E., et al., “A new alpha-conotoxin which targets alpha3beta2 nicotinic acetylcholine receptors,” J. Biol. Chem., 271: 7522-7528 (1996).
Charpantier, E., et al., “Nicotinic acetylcholine subunit mRNA expression in dopaminergic neurons of the rat substantia nigra and ventral tegmental area,” Neuroreport, 9(13): 3097-101 (1998).
Champtiaux, N., et al., “Distribution and pharmacology of alpha 6-containing nicotinic acetylcholine receptors analyzed with mutant mice,” J. Neurosci., 22: 1208-1217 (2002).
Chen, M., et al., “Nicotinic synapses formed between chick ciliary ganglion neurons in culture resemble those present on the neurons in vivo,” J. Neurobiol., 47(4): 265-79 (2001).
Cooper, E., et al., “Pentameric structure and subunit stoichiometry of a neuronal nicotinic acetylcholine receptor,” Nature (Lond) 350: 235-238 (1991).
Corrigall, W. A., “Nicotine self-administration in animals as a dependence model,” Nicotine Tob. Res., 1: 11-20 (1999).
Dani, J. A., “Overview of nicotinic receptors and their roles in the central nervous system,” Biol. Psychiatry, 49: 166-174 (2001).
Decker, M. W., et al., “Erysodine, a competitive antagonist at neuronal nicotinic acetylcholine receptors,” Eur. J. Pharmacol., 280: 79-89 (1995).
Di Chiara, G., “Role of dopamine in the behavioural actions of nicotine related to addiction,” Eur. J. Pharmacol., 393: 295-314 (2000).
Dunckley, T. and R. J. Lukas, “Nicotine modulates the expression of a diverse et of genes in the neuronal SH-SY5Y cell line,” J. Biol. Chem., 278(18):15633-15640 (2003).
Dutton, J. L. and D. J. Craik, “Alpha-Conotoxins: nicotinic acetylcholine receptor antagonists as pharmacological tools and potential drug leads,” Curr. Med. Chem., 84: 327-44 (2001).
Elliott, K. J., et al., “6-hydroxydopamine lesion of rat nigrostriatal dopaminergic neurons differentially affects nicotinic acetylcholine receptor subunit mRNA expression,” J. Mol. Neurosci., 10(3): 251-60 (1998).
Evans, N. M., et al., “Expression and functional characterisation of a human chimeric nicotinic receptor with alpha6beta4 properties,” Eur. J. Pharmacol., 466(1-2): 31-39 (2003).
Fu, Y., et al., “Inhibition of nicotine-induced hippocampal norepinephrine release in rats by alpha-conotoxins MII and AuIB microinjected into the locus coeruleus,” Neurosci. Lett., 266(2): 113-16 (1999).
Fucile, S., et al., “The neuronal alpha6 subunit forms functional heteromeric acetylcholine receptors in human transfected cells,” Eur. J. Neurosci., 10(1): 172-78 (1998).
Gerzanich, V., et al., “alpha5 subunit alters desensitization, pharmacology, Ca++ permeability and Ca++ modulation of human neuronal α3 nicotinic receptors,” J. Pharmacol. Exp. Ther., 286: 311-320 (1998).
Gerzanich, V., et al., “‘Orphan’ alpha6 nicotinic AChR subunit can form a functional heteromeric acetylcholine receptor,” Mol. Pharmacol., 51(2): 320-27 (1997).
Goldner, F. M., “Immunohistochemical localization of the nicotinic acetylcholine receptor subunit alpha6 to dopaminergic neurons in the substantia nigra and ventral tegmental area,” Neuroreport 8(12): 2739-42 (1997).
Grady, S. R., et al., “Nicotinic agonists stimulate acetylcholine release from mouse interpeduncular nucleus: a function mediated by a different nAChR than dopamine release from striatum,” J. Neurochem., 76(1): 258-68 (2001).
Grady, S. R., et al. “Characterization of nicotinic agonist-induced [(3)H]dopamine release from synaptosomes prepared from four mouse brain regions,” J. Pharmacol. Exp. Ther., 301(2) 651-60 (2002).
Guan, Z. Z., et al., “Selective changes in the levels of nicotinic acetylcholine receptor protein and of corresponding mRNA species in the brains of patients with Parkinson's disease,” Brain Res. 956(2): 358-66 (2002).
Guo, J. Z. and V. A. Chiappinelli, “A novel choline-sensitive nicotinic receptor subtype that mediates enhanced GABA release in the chick ventral lateral geniculate nucleus,” Neuroscience, 110(3): 505-13 (2002).
Han, Z. Y., et al., “Localization of nAChR subunit mRNAs in the brain of Macaca mulatta,” Eur. J. Neurosci., 12(10): 3664-74 (2000).
Harvey, S. C., et al., “Determinants of specificity for alpha-conotoxin MII on alpha3beta2 neuronal nicotinic receptors,” Mol. Pharmacol., 51(2): 336-42 (1997).
Itier, V. and D. Bertrand, “Neuronal nicotinic receptors: from protein structure to function,” FEBS Lett. 504: 118-125 (2001).
Jeyarasasingam, G., et al., “Stimulation of non-alpha7 nicotinic receptors partially protects dopaminergic neurons from 1-methyl-4-phenylpyridinium-induced toxicity in culture,” Neuroscience 109(2): 275-85 (2002).
Kaiser, S. A., et al., “Differential inhibition by alpha-conotoxin-MII of the nicotinic stimulation of [3H]dopamine release from rat striatal synaptosomes and slices,” J. Neurochem, 70(3): 1069-76 (1998).
Klink, R., et al., “Molecular and physiological diversity of nicotinic acetylcholine receptors in the midbrain dopaminergic nuclei., J. Neurosci., 21: 1452-1463 (2001).
Kulak, J. M., et al., “Loss of nicotinic receptors in monkey striatum after 1-methyl-4-phenyl-1,2,3,6-tetrahydropyridine treatment is due to a decline in α-conotoxin MII sites,” Mol. Pharmacol., 61: 230-238 (2002).
Kulak, J. M., et al., “Nicotine-evoked transmitter release from synaptosomes: functional association of specific presynaptic acetylcholine receptors and voltage-gated calcium channels,” J. Neurochem., 77(6): 1581-89 (2001).
Kulak, J. M., et al., “Declines in different beta2* nicotinic receptor populations in monkey striatum after nigrostriatal damage,” J. Pharmacol. Exp. Ther., 303(2): 633-39 (2002).
Kulak, J. M., et al., “5-Iodo-A-85380 binds to alpha-conotoxin MII-sensitive nicotinic acetylcholine receptors (nAChRs) as well as alpha4beta2* subtypes,” J. Neurochem., 81(2): 403-06 (2002).
Kuryatov, A., et al., “Human alpha6 AChR subtypes: subunit composition, assembly, and pharmacological responses,” Neuropharmacology, 39: 2570-2590 (2000).
Le Novere, N., “Involvement of alpha6 nicotinic receptor subunit in nicotine-elicited locomotion, demonstrated by in vivo antisense oligonucleotide infusion,” Neuroreport, 10: 2497-2501 (1999).
Le Novere, N., et al., “The diversity of subunit composition in nAChRs: evolutionary origins, physiologic and pharmacologic consequences,” J. Neurobiol., 53(4): 447-56 (2002).
Le Novere, N., et al., “Neuronal nicotinic receptor alpha 6 subunit mRNA is selectively concentrated in catecholaminergic nuclei of the rat brain,” Eur. J. Neurosci., 8(11): 2428-39 (1996).
Lena, C., et al., “Diversity and distribution of nicotinic acetylcholine receptors in the locus ceruleus neurons,” Proc. Natl. Acad. Sci. U.S.A, 96(21): 12126-31 (1999).
Louis, M. and P. B. Clarke, “Effect of ventral tegmental 6-hydroxydopamine lesions on the locomotor stimulant action of nicotine in rats,” Neuropharmacology, 37: 1503-1513 (1998).

Lukas, R. J., et al., “Some Methods of Studies of Nicotinic Acetylcholine Receptor Pharmacology,” Chapter 1, in Methods & New Frontiers in Neuroscience, CRC Press LLC, publisher (2002).

Marks, M. J., et al., “Differential agonist inhibition identifies multiple epibatidine binding sites in mouse brain,” J. Pharmacol. Exp. Ther., 285: 377-386 (1998).
Martin-Ruiz, C., et al., “Nicotinic receptors in the putamen of patients with dementia with Lewy bodies and Parkinson's disease: relation to changes in alpha-synuclein expression,” Neurosci. Lett., 335(2): 134-38 (2002).
Martin-Ruiz, C. M., et al., “Alpha and beta nicotinic acetylcholine receptors subunits and synaptophysin in putamen from Parkinson's disease,” Neuropharmacology, 39(13): 2830-39 (2000).
Marubio, L. M., et al., “Effects of nicotine in the dopaminergic system of mice lacking the alpha4 subunit of neuronal nicotinic acetylcholine receptors,” Eur. J. Neurosci., 17(7): 1329-37 (2003).
McCormick, D. A. and D. Contreras, “On the cellular and network bases of epileptic seizures,” Annu. Rev. Physiol., 63: 815-846 (2001).
McIntosh, J. M., et al., “Conus peptides: novel probes for nicotinic acetylcholine receptor structure and function,” Eur. J. Pharmacol, 393(1-3): 205-08 (2000).
Mogg, A. J., et al., “Methyllycaconitine is a potent antagonist of alpha-conotoxin-MII-sensitive presynaptic nicotinic acetylcholine receptors in rat striatum,” J. Pharmacol. Exp. Ther., 302(1): 197-204 (2002).
Murray, A. M., et al., “Damage to dopamine systems differs between Parkinson's disease and Alzheimer's disease with parkinsonism,” Annals of Neurology, 37(3): 300-312 (1995).
Museo, E. and R. A. Wise, “Locomotion induced by ventral tegmental microinjections of a nicotinic agonist,” Pharmacol., Biochem. Behav., 35: 735-737 (1990).
Nai, Q., et al., “Relating neuronal nicotinic acetylcholine receptor subtypes defined by subunit composition and channel function,” Mol. Pharmacol., 63(2): 311-24 (2003).
Nayak, S. V., et al., “Ca(2+) changes induced by different presynaptic nicotinic receptors in separate populations of individual striatal nerve terminals,” J. Neurochem., 76(6): 1860-70 (2001).

Penn, A. A., et al., “Competition in retinogeniculate patterning driven by spontaneous activity,” Science, 279(5359): 2108-12 (1998).
Pidoplichko, V. I., et al., “Nicotine activates and desensitizes midbrain dopamine neurons,” Nature (Lond), 390: 401-404 (1997).
Quik, M., et al. “Differential nicotinic receptor expression in monkey basal ganglia: effects of nigrostriatal damage,” Neuroscience 112(3): 619-30 (2002).
Quik, M., et al. “Differential declines in striatal nicotinic receptor subtype function after nigrostriatal damage in mice,” Mol. Pharmacol., 63(5): 1169-79 (2003).
Quik, M. and G. Jeyarasasingam, “Nicotinic receptors and Parkinson's disease,” Eur. J. Pharmacol., 393(1-3): 223-30 (2000).
Quik, M., et al., “Localization of nicotinic receptor subunit mRNAs in monkey brain by in situ hybridization,” J. Comp Neurol., 425(1): 58-69 (2000).
Quik, M., et al., “Vulnerability of 125I-alpha-conotoxin MII binding sites to nigrostriatal damage in monkey,” J. Neurosci., 21(15): 5494-500 (2001).
Quik, M., et al. “Differential nicotinic receptor expression in monkey basal ganglia: effects of nigrostriatal damage,” Neuroscience, 112(3): 619-30 (2002).
Rajput, A. and A. H. Rajput, “Progressive supranuclear palsy: Clinical features, pathophysiology and management,” Drugs Aging, 18(12): 913-25 (2001).
Ramirez-Latorre, J., et al., “Functional contributions of alpha5 subunit to neuronal acetylcholine receptor channels,” Nature (Lond) 380: 347-351 (1996).
Ridley, D. L., et al., “Effects of chronic drug treatments on increases in intracellular calcium mediated by nicotinic acetylcholine receptors in SH-SY5Y cells,” Br. J. Pharmacol., 135(4): 1051-59 (2002).

Ross, R. A., et al., “Coordinate Morphological and Biochemical Interconversion of Human Neuroblastoma Cells,” JNCI, 71(4): 741-749 (1983).
Ross, S. A., et al., “Phenotypic characterization of an alpha 4 neuronal nicotinic acetylcholine receptor subunit knock-out mouse,” J. Neurosci., 20: 6431-6441 (2000).
Rusted, J. M., et al., “Nicotinic treatment for degenerative neuropsychiatric disorders such as Alzheimer's disease and Parkinson's disease,” Behav. Brain Res., 113(1-2): 121-29 (2000).
Sabbagh, M. N., et al., “Correlation of nicotinic receptor binding with clinical and neuropathological changes in Alzheimer's disease and dementia with Lewy bodies,” J. Neural Transm., 108(10): 1149-57 (2001).
Schneider, J. S., et al., “Effects of SIB-1508Y, a novel neuronal nicotinic acetylcholine receptor agonist, on motor behavior in parkinsonian monkeys,” Mov. Disord., 13(4): 637-42 (1998).
Schneider, J. S., et al. “Effects of the nicotinic acetylcholine receptor agonist SIB-1508Y on object retrieval performance in MPTP-treated monkeys: comparison with levodopa treatment,” Ann. Neurol., 43(3): 311-17 (1998).
Sharples, C. G., et al., “UB-165: a novel nicotinic agonist with subtype selectivity implicates the alpha4beta2* subtype in the modulation of dopamine release from rat striatal synaptosomes,” J. Neurosci., 20(8): 2783-91 (2000).
Shon, K. J., et al., “Three-dimensional solution structure of alpha-conotoxin MII, an alpha3beta2 neuronal nicotinic acetylcholine receptor-targeted ligand,” Biochemistry, 36(50): 15693-700 (1997).

Stitzel, J. A., et al., “Sensitivity to the seizure-inducing effects of nicotine is associated with strain-specific variants of the α5 and α7 nicotinic receptor subunit genes,” J. Pharmacol. Exp. Ther., 284: 1104-1111 (1998).
Stitzel, J. A., et al., “Potential role of the α4 and α6 nicotinic receptor subunits in regulating nicotine-induced seizures,” J. Pharmacol. Exp. Ther., 293: 67-74 (2000).

Tavazoie, S. F., et al., “Differential block of nicotinic synapses on B versus C neurones in sympathetic ganglia of frog by alpha-conotoxins MII and ImI,” Br. J. Pharmacol., 120(6): 995-1000 (1997).

Vailati, S., et al., “Functional alpha6-containing nicotinic receptors are present in chick retina,” Mol. Pharmacol., 56(1): 11-19 (1999).
Vailati, S., et al., “beta3 subunit is present in different nicotinic receptor subtypes in chick retina,” Eur. J. Pharmacol., 393(1-3): 23-30 (2000).
Wada, E., et al., “The distribution of mRNA encoded by a new member of the neuronal nicotinic acetylcholine receptor gene family (alpha 5) in the rat central nervous system,” Brain Res., 526: 45-53 (1990).
Wada, E., et al., “Distribution of alpha 2, alpha 3, alpha 4 and beta 2 neuronal nicotinic receptor subunit mRNAs in the central nervous system: a hybridization histochemical study in the rat,” J. Comp. Neurol., 284: 314-335 (1989).
Wang, F., et al., “Assembly of human neuronal nicotinic receptor α5 subunits with α3, β2 and β4 subunits,” J. Biol. Chem., 271: 17656-17665 (1996).
Wang, N., et al., “Autonomic function in mice lacking alpha5 neuronal nicotinic acetylcholine receptor subunit,” J. Physiol., 542: 347-354 (2002).
Whiteaker, P., et al., “Identification of a novel nicotinic binding site in mouse brain using [125I]-epibatidine,” Br. J. Pharmacol., 131: 729-739 (2000a).
Whiteaker, P., et al., “¹²⁵I-Alpha-conotoxin MII identifies a novel nicotinic acetylcholine receptor population in mouse brain,” Mol. Pharmacol., 57: 913-925 (2000b).
Whiteaker, P., et al., “Pharmacological and null mutation approaches reveal nicotinic receptor diversity,” Eur. J. Pharmacol., 393: 123-135 (2000c).
Yu, C. R. and L. W. Role, “Functional contribution of the alpha5 subunit to neuronal nicotinic channels expressed by chick sympathetic ganglion neurons,” J. Physiol. (Lond), 509: 667-681 (1998).
Zoli, M., et al., “Identification of four classes of brain nicotinic receptors using beta 2 mutant mice,” J. Neurosci., 18: 4461-4472 (1998).
Zoli, M., et al. “Identification of the nicotinic receptor subtypes expressed on dopaminergic terminals in the rat striatum,” J. Neurosci., 22(20): 8785-89 (2002).

The foregoing are hereby fully incorporated by reference.

### REFERENCED U.S. PATENT DOCUMENTS

In addition, the following U.S. patents and International Patent Applications are hereby fully incorporated herein by reference: U.S. Pat. Nos. 5,369,028; 5,801,232; 5,837,489; 5,981,193; 6,440,681; 6,485,967; and 6,524,789.

What is claimed is:

1. A cultured eukaryotic cell transfected with one or more isolated nucleic acid molecules comprising a sequence or sequences of nucleotides or ribonucleotides that encode α6, β3, β4, and α5 subunits of a nicotinic acetylcholine receptor (nAChR).

2. The cultured eukaryotic cell of claim 1, wherein the one or more isolated nucleic acid molecules are contained within an expression vector or vectors.

3. The cultured eukaryotic cell of claim 1, wherein the cell is cultured under conditions that lead to cell surface expression of a functional nAChR comprising at least one each of α6, β3, β4, and α5 subunits.

4. The cultured eukaryotic cell of claim 1, wherein the cell is derived from a human neuroblastoma cell line.

5. A method of making cultured eukaryotic cells of claim 1 having nicotinic acetylcholine receptor (nAChR) activity, comprising:
a) introducing one or more isolated nucleic acid molecules that encode the α6, β3, β4, and α5 subunits of a nAChR into eukaryotic cells;
b) selecting cells from a) that express detectable α6, β3, β4, and α5 subunit proteins; and
c) detecting nAChR activity in the selected cells,
wherein the activity is mediated by a receptor containing at least one each of the α6, β3, β4, and α5 subunits encoded by the isolated one or more nucleic acid molecules.

6. A kit useful for making cultured eukaryotic cells of claim 1, comprising
a) a container or containers comprising one or more isolated nucleic acid molecules that encode at least one each of the α6, β3, β4, and α5 subunits of a nAChR;
b) a container comprising suitable eukaryotic cells; and
c) instructions for the transfection of the nucleic acids into the cells and for achieving culture conditions favoring expression of functional nAChR reporters containing at least one each of the receptor subunits encoded by the nucleic acid molecules.

7. A method for identifying compounds that are antagonists, partial agonists, or agonists of nicotinic acetylcholine receptors (nAChRs), said method comprising:
a) contacting recombinant cells with a test compound, wherein:
i) the recombinant cells are produced by transfection of suitable eukaryotic cells with nucleic acid encoding at least one each of α6, β3, β4, and α5 nAChR subunits;
ii) the recombinant cells express an nAChR comprising at least one each of the nAChR subunits encoded by the transfected nucleic acid; and
iii) the expressed nAChR subunits form nAChR comprising at least one each of α6, β3, β4, and α5 nAChR subunits; and
b) measuring ion flux, the electrophysiological response of the cells, or binding of the test compound to the nAChR, whereby antagonists, partial agonists, or agonists of the nAChR are identified.

8. The method of claim 7, wherein binding of the test compound to the nAChR is used initially to select compounds for further testing.

9. The method of claim 7, further comprising comparing an effect of the test compound according to a) and b) to the effect of the test compound according to a) and b) on cells that are substantially identical to the recombinant cells but have not been transfected with nucleic acid encoding the nAChR subunits and that do not express the nAChR.

10. The method of claim 7, further comprising comparing the effect of the test compound on ion flux or electrophysiological response of the cells to ion flux or electrophysiological response of the cells in the absence of the test compound.

11. The method of claim 7, wherein the recombinant cells further comprise a nucleic acid comprising a reporter gene encoding a detectable gene product selected from the group consisting of mRNA and a polypeptide, operatively linked to nucleic acid encoding a transcriptional control element wherein the activity of the transcriptional control element is regulated by the nAChR; and the interaction of the test compound with the nAChRs is measured by detecting the gene product encoded by the reporter gene.

12. The method of claim 11, wherein antagonism by the test compound is detected by exposing the test compound to the recombinant cells in the presence of a known agonist and measuring the reduction in the gene product encoded by the reporter gene.

13. A cultured eukaryotic cell transfected with one or more isolated nucleic acid molecules comprising a sequence or sequences of nucleotides or ribonucleotides that encode α6, α4, β2, and β3 subunits of a nicotinic acetylcholinergic receptor (nAChR).

14. The cultured eukaryotic cell of claim 13, wherein the isolated one or more nucleic acid molecules are contained within an expression vector or vectors.

15. The cultured eukaryotic cell of claim 13, wherein the cell is cultured under conditions that lead to cell surface expression of a functional nAChR comprising at least one each of α6, α4, β2, and β3 subunits.

16. The cultured eukaryotic cell of claim 13, wherein the cell is derived from a human neuroblastoma cell line.

17. A method of making cultured eukaryotic cells of claim 13 having nicotinic acetylcholine receptor (nAChR) activity, comprising:
a) introducing one or more isolated nucleic acid molecules that encode the α6, α4, β2, and β3 subunits of a nAChR into eukaryotic cells;
b) selecting cells from a) that express detectable α6, α4, β2, and β3 subunit proteins; and
c) detecting nAChR activity in the selected cells,
wherein the activity is mediated by a receptor containing at least one each of the α6, α4, β2, and β3 subunits encoded by the isolated one or more nucleic acid molecules.

18. A kit useful for making cultured eukaryotic cells of claim 13, comprising
a) a container or containers comprising one or more isolated nucleic acid molecules that encode at least one each of the α6, α4, β2, and β3 subunits of a nAChR;
b) a container comprising suitable eukaryotic cells; and
c) instructions for the transfection of the nucleic acids into the cells and for achieving culture conditions favoring expression of functional nAChR reporters containing at least one each of the receptor subunits encoded by the nucleic acid molecules.

19. A method for identifying compounds that are antagonists, partial agonists, or agonists of nicotinic acetylcholine receptors (nAChRs), said method comprising:
a) contacting recombinant cells with a test compound, wherein:
i) the recombinant cells are produced by transfection of suitable eukaryotic cells with nucleic acid encoding at least one each of α6, α4, β2, and β3 nAChR subunits;
ii) the recombinant cells express an nAChR comprising at least one each of the nAChR subunits encoded by the transfected nucleic acid; and
iii) the expressed nAChR subunits form nAChR comprising at least one each of α6, α4, β2, and β3 nAChR subunits; and
b) measuring ion flux, the electrophysiological response of the cells, or binding of the test compound to the nAChR, whereby antagonists, partial agonists, or agonists of the nAChR are identified.

20. The method of claim 19, wherein binding of the test compound to the nAChR is used initially to select compounds for further testing.

21. The method of claim 19, further comprising comparing an effect of the test compound according to a) and b) to the effect of the test compound according to a) and b) on cells that are substantially identical to the recombinant cells but have not been transfected with nucleic acid encoding the nAChR subunits and that do not express the nAChR.

22. The method of claim 19, further comprising comparing the effect of the test compound on ion flux or electrophysiological response of the cells to ion flux or electrophysiological response of the cells in the absence of the test compound.

23. The method of claim 19, wherein the recombinant cells further comprise a nucleic acid comprising a reporter gene encoding a detectable gene product selected from the group consisting of mRNA and a polypeptide, operatively linked to nucleic acid encoding a transcriptional control element wherein the activity of the transcriptional control element is regulated by the nAChR; and the interaction of the test compound with the nAChRs is measured by detecting the gene product encoded by the reporter gene.

24. The method of claim 23, wherein antagonism by the test compound is detected by exposing the test compound to the recombinant cells in the presence of a known agonist and measuring the reduction in the gene product encoded by the reporter gene.
